(19) United States
(12) Patent Application Publication
(10) Pub. No.: US 2006/0140965 A1
(43) Pub. Date: Jun. 29, 2006

(54) Immunogenic compositions comprising a xenogenic prostate protein p501s

(76) Inventors: Jean-Pol Cassart, Rixensart (BE); Catherine Marie Ghislaine Gerard, Rixensart (BE); Paul A Hamblin, Stevenage (GB); Remi M Palmantier, Rixensart (BE)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/517,552

(22) PCT Filed: Jun. 6, 2003
(86) PCT No.: PCT/EP03/06095

§ 371 Date: Sep. 16, 2005

(30) Foreign Application Priority Data
Jun. 11, 2002 (GB) 0213364.3
Sep. 18, 2002 (GB) 0221689.3

(51) Int. Cl. A61K 48/00; A61K 39/00

(52) U.S. Cl. 424/185.1; 514/44

(57) ABSTRACT

The present invention relates to pharmaceutical/immunogenic compositions and methods for inducing an immune response against tumour-related antigens. More specifically, the invention relates to non-human prostate-specific antigens, more precisely to the non-human prostate-specific P501S, which can be used as xenogeneic antigen in prostate cancer vaccine therapy and as diagnostic agents for prostate tumours in humans, to immunogenic compositions containing them, to methods of manufacture of such compositions and to their use in medicine. Methods for formulating vaccines for immunotherapeutically treating P501S-expressing prostate tumors, prostatic hyperplasia, and prostate intraepithelilial neoplasia (PIN) are also provided.

The present invention relates to immunogenic compositions and methods for inducing an immune response against tumours-related antigens. More specifically, the invention relates to non-human prostate-specific antigens which can be used as xenogeneic antigens to induce prostate-directed immunity in humans, to pharmaceutical compositions containing them, to methods of manufacture of such compositions and to their use in medicine. In particular the compositions of the invention include the prostate-specific protein known as P501S, from a non human origin. Such compositions find utility in cancer vaccine therapy, particularly prostate cancer vaccine therapy and diagnostic agents for prostate tumours. The present invention also provides methods for formulating vaccines for immunotherapeutically treating prostate cancer patients and P501S-expressing tumours other than prostate tumours, prostatic hyperplasia, and prostate intraepithelial neoplasia (PIN).

### BACKGROUND OF THE INVENTION

Prostate cancer is the most common cancer among males, with an estimated incidence of 30% in men over the age of 50. Overwhelming clinical evidence shows that human prostate cancer has the propency to metastasise to bone, and the disease appears to progress inevitably from androgen dependent to androgen refractory status, leading to increased patient mortality (Abbas F., Scardino P. “The Natural History of Clinical Prostate Carcinoma.” In Cancer (1997); 80:827-833). This prevalent disease is currently the second leading cause of cancer death among men in the US.

Despite considerable research into therapies for the disease, prostate cancer remains difficult to treat. Currently, treatment is based on surgery and/or radiation therapy, but these methods are ineffective in a significant percentage of cases (Frydenberg M., Stricker P., Kaye K. “Prostate Cancer Diagnosis and Management” The Lancet (1997); 349:1681-1687). Several tumour-associated antigens are already known. Many of these antigens may be interesting targets for immunotherapy, but are either not fully tumour-specific or are closely related to normal proteins, and hence bear with them the risk of organ-specific auto-immunity, once targeted by a potent immune response. When an auto-immune response to non-crucial organs can be tolerated, auto-immunity to heart, intestine and other crucial organs could lead to unacceptable safety profiles. Some previously identified prostate specific proteins like prostate specific antigen (PSA) and prostatic acid phosphatase (PAP), prostate-specific membrane antigen (PSMA) and prostate stem cell antigen (PSCA) used in vaccine preparations have only showed limited therapeutic potential so far (Pound C., Partin A., Eisenberg M. et al. “Natural History of Progression after PSA Elevation following Radical Prostatectomy.” In Jama (1999); 281:1591-1597) (Bostwick D., Pacelli A., Blute M. et al. “Prostate Specific Membrane Antigen Expression in Prostatic Intraepithelial Neoplasia and Adenocarcinoma.” In Cancer (1998); 82:2256-2261), and this limitation may be due to a relatively poor immunogenicity due to their self nature, or by poor prostate and tumour-specificity.

The existence of tumour rejection mechanisms has been recognised since several decades. Tumour antigens, though encoded by the genome of the organism and thus theoretically not recognized by the immune system through the immune tolerance phenomenon, can occasionally induce immune responses detectable in cancer patients. This is evidenced by antibodies or T cell responses to antigens expressed by the tumour (Xue B H., Zhang Y., Sosman J. et al. “Induction of Human Cytotoxic T-Lymphocytes Specific for Prostate-Specific Antigen.” In Prostate (1997); 30(2):73-78). When relatively weak anti-tumour effects can be observed through the administration of antibodies recognizing cell surface markers of tumour cells, induction of strong T cell responses to antigens expressed by tumour cells can lead to complete regression of established tumours in animal models (mainly murine).

It is now recognised that the expression of tumour antigens by a cell is not sufficient for induction of an immune response to these antigens. Initiation of a tumour rejection response requires a series of immune amplification phenomena dependent on the intervention of antigen presenting cells, responsible for delivery of a series of activation signals.

Human P501S is a membrane protein which interacts with a cell surface receptor. It is predicted to be a type IIIa plasma membrane protein with 9-11 transmembrane regions spanning the whole length of the protein. P501S shares some homologies with spinash sucrose binding protein (Riesmeier J W, Willmitzer L, Frommer W B, 1992, EMBO J. 11, 4705-13). Human P501S as described in WO 98/37418, and its C-terminal fragments PS108 as described in WO 98/50567 and Y54369 as described in WO 99/67384, is a human prostate specific antigen, associated with a prostate tissue disease or condition, especially with prostate cancer. Its expression is observed in normal and tumour prostate tissue as well as in some breast metastasis (WO 00/61756).

P501S nucleotide sequence and deduced polypeptide sequence and fragments are disclosed in WO 98/37418. Contiguous and partially overlapping cDNA fragments and polypeptides encoded thereby, have also been described (WO 98/50567), more particularly a C-terminal fragment of 255 amino acids in length. A polypeptide of 231 amino acids in length, described in WO 99/67384, is reported to comprise a potential transmembrane domain, two potential caseine kinase II phosphorylation sites, one potential protein kinase C phosphorylation site and a potential cell attachment sequence.

P501S is described as being a member of the family of human “self” antigens”, against which it will be suposedly difficult to induce an “auto-immune” response, including CD8+ cytotoxic T-lymphocyte (CTL) responses. Therefore efficient vaccine strategies directed against P501S will require the development of methods to overcome the immune tolerance to the self-protein.

The present invention is concerned with an efficient antigen-specific immunotherapy of human malignancies, more especially of human prostate cancer. It takes advantage of the surprising observation that humans immunised with an antigen from a xenogeneic (non human) origin, are capable of mounting a effective immune response against the human antigen counterpart, through the generation of cross-reactive antibodies and/or T cells. Such an approach has the advantages over classical immunotherapy that utilises human prostate self antigens, since these antigens are tolerated by the human body and it is therefore difficult to raise an immune response against the antigen (Fong et al, J. Immunol., 1997, 156:3313-3117; Fong et al, J. Immunol., 2001, 167:7150-7156)

### STATEMENT OF THE INVENTION

Accordingly, the present invention provides for pharmaceutical/immunogenic compositions comprising a xenogeneic P501S polypeptide or a xenogeneic P501S-encoding polynucleotide, or an immunogenic fragment thereof; and a pharmaceutically acceptable carrier. Preferably the xenogeneic P501S polypeptide is selected from the group comprising SEQ ID NO:1 or SEQ ID NO:3 or SEQ ID NO:10 and the xenogeneic P501S-encoding polynucleotide is selected from the group comprising SEQ ID NO:2 or SEQ ID NO:4 or SEQ ID NO:11. Preferably the compositions comprises a TH-1 adjuvant.

The invention also provides for immunogenic compositions comprising an effective amount of antigen presenting cells, modified by in vitro loading with a xenogeneic P501S polypeptide or immunogenic fragment thereof, or genetically modified in vitro to express a xenogeneic P501S polypeptide and a pharmaceutically effective carrier.

In another embodiment the invention relates to an isolated polypeptide comprising an amino acid sequence which has at least 92% identity to the amino acid sequence of SEQ ID NO:1 over the entire length of of SEQ ID NO:1; to a polynucleotide encoding said polypeptide, and to expression vectors or a recombinant live microorganisms comprising said polynucleotide.

Also provided is a process for the production of an immunogenic composition as herein described, comprising admixing a xenogeneic P501S polypeptide or a xenogeneic P501S-encoding polynucleotide with a suitable adjuvant, diluent or other pharmaceutically acceptable carrier.

The present invention also provides methods for purifying the xenogeneic P501S antigens and for formulating immunogenic compositions for immunotherapeutically treating P501S-expressing prostate tumors, prostatic hyperplasia and prostate intraepithelilial neoplasia (PIN).

The present invention also provides pharmaceutical/immunogenic compositions and vaccine compositions suitable for use in medicine, and more especially in the treatment of a prostate tumours, said composition comprising a xenogeneic P501S antigen. More particularly, the invention is directed to a mouse, rat and monkey P501S which can be used as a xenogeneic form of human P501S antigen to induce prostate-targeted immunity in humans.

The invention further relates to the use of a polypeptide or a polynucleotide as herein described in the manufacture of a vaccine for immunotherapeutically treating a patient suffering from or susceptible to prostate cancer or other P501S-associated tumours or diseases.

In another embodiment the invention also relates to a method of inducing an immune response against human P501S in a human, comprising administering to the subject an effective dosage of a pharmaceutical or immunogenic composition comprising a xenogeneic form of said human P501S. Preferably, the composition includes a live viral expression system or a plasmid vector which expresses said xenogeneic antigen, ot through antigen loaded dendritic cells.

### DETAILED DESCRIPTION OF THE INVENTION

A xenogeneic form of antigen refers to an antigen having substantial sequence identity to the human antigen (also termed autologous antigen) which serves as a reference antigen but which is derived from a different non-human species. In this context the substantial identity refers to concordance of an amino acid sequence with another amino acid sequence or of a polynucleotide sequence with another polynucleotide sequence when such sequence are arranged in a best fit alignment in any of a number of sequence alignment proteins known in the art. By substantial identity is meant at least 70-98%, and preferably at least 85-95% sequence identity between the compared sequences. Therefore according to the invention the xenogeneic P501S will be a P501S polypeptide which is xenogeneic with respect to human P501S, in other words which is isolated from a species other than human. In a preferred embodiment, the polypeptide is isolated from mouse, rat, or Cynomolgus monkey (Maccaca fascicularis). In a more preferred embodiment, the P501S polypeptide has the sequence set forth in SEQ ID NO:1 (rat), in SEQ ID NO:3 (Cynomolgus monkey) or in SEQ ID NO:10 (mouse). The isolated xenogeneic P501S polypeptide will generally share substantial sequence similarity, and include isolated polypeptides comprising an amino acid sequence which has at least 70% identity, preferably at least 80% identity, more preferably at least 90% identity, yet more preferably at least 95% identity, most preferably at least 97-99% identity, to that of SEQ ID NO:1, SEQ ID NO:3 or SEQ ID NO:10 over the entire length of SEQ ID NO:1, SEQ ID NO:3 or SEQ ID NO:10 respectively. Accordingly the polypeptide will comprise an immunogenic fragment of the polypeptide SEQ ID NO:1, SEQ ID NO:3 or SEQ ID NO:10 in which the immunogenic activity of the immunogenic fragment is substantially the same as the polypeptide SEQ ID NO:1, SEQ ID NO:3 or SEQ ID NO:10 respectively. The polypeptide sequence as set forth in SEQ ID NO:1 and the polynucleotide sequence as set forth in SEQ ID NO:2 are novel and also form part of the invention. In particular the invention provides an isolated polypeptide comprising an amino acid sequence which has at least 90%, preferably at least 92% identity to the amino acid sequence of SEQ ID NO:1 over the entire length of of SEQ ID NO:1. Preferably the isolated polypeptide amino acid sequence has at least 95% identity to SEQ ID NO:1. Still more preferably the polypeptide comprises the amino acid sequence of SEQ ID NO:1. Most preferably the polypeptide is the isolated polypeptide of SEQ ID NO:1.

In addition the polypeptide can be a fragment of at least about 20 consecutive amino acids, preferably about 30, more preferably about 50, yet more preferably about 100, most preferably about 150 contiguous amino acids selected from the amino acid sequences as shown in SEQ ID NO:1, SEQ ID NO:3 or SEQ ID NO:10. More particularly fragments will retain some functional property, preferably an immunological activity, of the larger molecule set forth in SEQ ID NO:1, SEQ ID NO:3 or SEQ ID NO:10, and are useful in the methods described herein (e.g. in pharmaceutical, immunogenic and vaccine compositions, in diagnostics, etc.). In particular the fragments will be able to generate an immune response against the human counterpart, such as the generation of cross-reactive antibodies which react with the autologous human form of P501S as set forth in any of the SEQ ID NO: 5 (Corixa WO 98/37418), SEQ ID NO: 6 (Abbott WO 98/50567) and SEQ ID NO:7 (Incyte WO 99/67384).

In one embodiment, the polypeptide of the invention may be part of a larger fusion, comprising the tumour-associated xenogeneic P501S or fragment thereof and a heterologous protein or part of a protein acting as a fusion partner. The protein and the fusion partner may be chemically conjugated, but are preferably expressed as recombinant fusion proteins in a heterologous expression system. In a preferred embodiment of the invention there is provided a xenogeneic P501S fusion protein linked to an immunological fusion partner that may provides additional T helper epitopes thereby further assisting in breaking the tolerance against the autologous antigen. Thus the fusion partner may act through a bystander helper effect linked to secretion of activation signals by a large number of T cells specific to the foreign protein or peptide, thereby enhancing the induction of immunity to the P501S component as compared to the non-fused xenogeneic protein. Preferably the heterologous partner is selected to be recognizable by T cells in a majority of humans.

In another embodiment, the invention provides a xenogeneic P501S protein or fragment or homologues thereof linked to a fusion partner that acts as an expression enhancer. Thus the fusion partner may assist in aiding in the expression of P501S in a heterologous system, allowing increased levels to be produced in an expression system as compared to the native recombinant protein.

Preferably the fusion partner will be both an immunological fusion partner and an expression enhancer partner thereby assisting in aiding the expressing and in breaking the tolerance against the autologous antigen. Accordingly, the present invention in the embodiment provides fusion proteins comprising the tumour-specific P501S or a fragment thereof linked to a fusion partner. Preferably the fusion partner is acting both as an immunological fusion partner and as an expression enhancer partner. Accordingly, in a preferred form of the invention, the fusion partner is the non-structural protein from influenzae virus, NS1 (hemagglutinin) or fragment thereof. Typically the N-terminal 81 amino acids are utilised, although different fragments may be used provided they include T-helper epitopes (C. Hackett, D. Horowitz, M. Wysocka & S. Dillon, 1992, J. Gen. Virology, 73, 1339-1343). When NS1 is the immunological fusion partner it has the additional advantage in that it allows higher expression yields to be achieved. In particular, such fusions are expressed at higher yields than the native recombinant P501S proteins. In another preferred form of the invention, the immunological fusion partner is derived from protein D, a surface protein of the gram-negative bacterium, Haemophilus influenza B (WO91/18926). Preferably the protein D derivative comprises approximately the first 113 of the protein, in particular approximately the first N-terminal 100-110 amino acids. Preferably the protein D derivative is lipidated. Preferably the first 109 residues of the Lipoprotein D fusion partner is included on the N-terminus to provide the vaccine candidate antigen with additional exogenous T-cell epitopes and increase expression level in E-coli (thus acting also as an expression enhancer). The lipid tail ensures optimal presentation of the antigen to antigen presenting cells. In another embodiment the immunological fusion partner is the protein known as LYTA. Preferably the C terminal portion of the molecule is used. Lyta is derived from Streptococcus pneumoniae which synthesize an N-acetyl-L-alanine amidase, amidase LYTA, (coded by the lytA gene {Gene, 43 (1986) page 265-272} an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at its amino terminus has been described (Biotechnology: 1992, 10, 795-798). As used herein a preferred embodiment utilises the repeat portion of the Lyta molecule found in the C terminal end starting at residue 178. A particularly preferred form incorporates residues 188-305. In another preferred embodiment, the immunological fusion partner is derived from a Mycobacterium sp., such as a Mycobacterium tuberculosis-derived Ra12 fragment. Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example Skeiky et al., Infection and Immun. 1999, 67:3998-4007). C-terminal fragments of the MTB32A coding sequence express at high levels and remain as a soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide.

The proteins of the present invention are expressed in an appropriate host cell, and preferably in E. coli or in yeast such as in Pichia pastoris or Saccharomyces cerevisiae. In a preferred embodiment the proteins are expressed with an affinity tag, such as for example, a histidine tail comprising between 5 to 9 and preferably six histidine residues, most preferably at least 4 histidine residues. These are advantageous in aiding purification through for example ion metal affinity chromatography (IMAC).

The proteins of the present invention are provided either soluble in a liquid form or in a lyophilised form, which is the preferred form. It is generally expected that each human dose will comprise 1 to 1000 μg of protein, and preferably 30-300 μg.

The present invention also provides a nucleic acid encoding the proteins of the present invention. In a preferred embodiment, the xenogeneic P501S polynucleotide has the sequence set forth in SEQ ID NO:2 (rat) or in SEQ ID NO:4 (Cynomolgus monkey) or in SEQ ID NO:11 (mouse). The isolated xenogeneic P501S polynucleotides of the invention may be single-stranded (coding or antisense) or double-stranded, and may be DNA (genomic, cDNA or synthetic) or RNA molecules. Additional coding or non-coding sequences may, but need not, be present within a polynucleotide of the present invention. In other related embodiments, the present invention provides polynucleotide variants having substantial identity to the sequences disclosed herein in SEQ ID NO:2, in SEQ ID NO:4 or in SEQ ID NO:11, for example those comprising at least 70% sequence identity, preferably at least 75%, 80%, 85%, 90%, 95%, 96%, 97%, 98%, or 99% or higher, sequence identity compared to a polynucleotide sequence of this invention using the methods described herein, (e.g., BLAST analysis using standard parameters). In a related embodiment, the isolated polynucleotide of the invention will comprise a nucleotide sequence encoding a polypeptide that has at least 90%, preferably 95% and above, identity to the amino acid sequence of SEQ ID NO:1, in SEQ ID NO:3 or in SEQ ID NO:10 over the entire length of SEQ ID NO:1, in SEQ ID NO:3 or in SEQ ID NO:10 respectively; or a nucleotide sequence complementary to said isolated polynucleotide.

Such sequences can be inserted into a suitable expression vector and used for DNA/RNA vaccination or expressed in a suitable host. The expression vectors comprising the isolated polynucleotide sequence according to the invention, and the appropriate hosts also form part of the invention. In additional embodiments, genetic constructs comprising one or more of the polynucleotides of the invention are introduced into cells in vivo. This may be achieved using any of a variety or well-known approaches. One of the preferred methods for in vivo delivery of one or more nucleic acid sequences involves the use of an expression vector such as a recombinant live viral or bacterial microorganism. Suitable viral expression vectors are for example poxviruses (e.g; vaccinia, fowlpox, canarypox), alphaviruses (Sindbis virus, Semliki Forest Virus, Venezuelian Equine Encephalitis Virus), adenoviruses, adeno-associated virus, picornaviruses (poliovirus, rhinovirus), and herpesviruses (varicella zoster virus, etc). Other preferred methods for in vivo delivery of one or more nucleic acid sequences involves the use of a bacterial expression vector, such as Listeria, Salmonella, Shigella and BCG. Inoculation and in vivo infection with this live vector will lead to in vivo expression of the antigen and induction of immune responses. These viruses and bacteria can be virulent, or attenuated in various ways in order to obtain live vaccines. Such live vaccines also form part of the invention.

It is an embodiment of the invention that the antigens, including nucleic acid vector, of the invention be utilised with immunostimulatory agent. Preferably the immunostimulatory agent is administered at the same time as the antigens of the invention and in preferred embodiments are formulated together. Such immunostimulatory agents include but are not limited to: synthetic imidazoquinolines such as imiquimod [S-26308, R-837], (Harrison, et al., Vaccine 19: 1820-1826, 2001; and resiquimod [S-28463, R-848] (Vasilakos, et al., Cellular immunology 204: 64-74, 2000.; Schiff bases of carbonyls and amines that are constitutively expressed on antigen presenting cell and T-cell surfaces, such as tucaresol (Rhodes, J. et al., Nature 377: 71-75, 1995), cytokine, chemokine and co-stimulatory molecules as either protein or peptide, including for example pro-inflammatory cytokines such as Interferon, GM-CSF, IL-1 alpha, IL-1 beta, TGF-alpha and TGF-beta, Th1 inducers such as interferon gamma, IL-2, IL-12, IL-15, IL-18 and IL-21, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L, other immunostimulatory targeting ligands such as CTLA-4 and L-selectin, apoptosis stimulating proteins and peptides such as Fas, (49), synthetic lipid based adjuvants, such as vaxfectin, (Reyes et al., Vaccine 19: 3778-3786, 2001) squalene, alpha-tocopherol, polysorbate 80, DOPC and cholesterol, endotoxin, [LPS], (Beutler, B., Current Opinion in Microbiology 3: 23-30, 2000); CpG oligo- and di-nucleotides (Sato, Y. et al., Science 273 (5273): 352-354, 1996; Hemmi, H. et al., Nature 408: 740-745, 2000) and other potential ligands that trigger Toll receptors to produce Th1-inducing cytokines, such as synthetic Mycobacterial lipoproteins, Mycobacterial protein p19, peptidoglycan, teichoic acid and lipid A.

Other suitable adjuvant include CT (cholera toxin, subunites A and B) and LT (heat labile enterotoxin from E. coli, subunites A and B), heat shock protein family (HSPs), and LLO (listeriolysin O; WO 01/72329).

As will be understood by those of skill in the art, it may be advantageous in some instances to produce polypeptide-encoding nucleotide sequences possessing non-naturally occurring codons. The DNA code has 4 letters (A, T, C and G) and uses these to spell three letter “codons” which represent the amino acids the proteins encodes in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons.

Where more than one codon is available to code for a given amino acid, it has been observed that the codon usage patterns of organisms are highly non-random. Different species show a different bias in their codon selection and, furthermore, utilisation of codons may be markedly different in a single species between genes which are expressed at high and low levels. This bias is different in viruses, plants, bacteria and mammalian cells, and some species show a stronger bias away from a random codon selection than others. For example, humans and other mammals are less strongly biased than certain bacteria or viruses. For these reasons, there is a significant probability that a mammalian gene expressed in E. coli or a viral gene expressed in mammalian cells will have an inappropriate distribution of codons for efficient expression. It is believed that the presence in a heterologous DNA sequence of clusters of codons which are rarely observed in the host in which expression is to occur, is predictive of low heterologous expression levels in that host.

In consequence, codons preferred by a particular prokaryotic (for example E. coli or yeast) or eukaryotic host can be optimised, that is selected to increase the rate of protein expression, to produce a recombinant RNA transcript having desirable properties, such as for example a half-life which is longer than that of a transcript generated from the naturally occurring sequence, or to optimise the immune response in humans. The process of codon optimisation may include any sequence, generated either manually or by computer software, where some or all of the codons of the native sequence are modified. Several method have been published (Nakamura et. al., Nucleic Acids Research 1996, 24:214-215; WO98/34640). One preferred method according to this invention is Syngene method, a modification of Calcgene method (R. S. Hale and G Thompson (Protein Expression and Purification Vol. 12 pp. 185-188 (1998)). This process of codon optimisation may have some or all of the following benefits: 1) to improve expression of the gene product by replacing rare or infrequently used codons with more frequently used codons, 2) to remove or include restriction enzyme sites to facilitate downstream cloning and 3) to reduce the potential for homologous recombination between the insert sequence in the DNA vector and genomic sequences and 4) to improve the immune response in humans. Due to the nature of the algorithms used by the SynGene programme to generate a codon optimised sequence, it is possible to generate an extremely large number of different codon optimised sequences which will perform a similar function. In brief, the codons are assigned using a statistical method to give synthetic gene having a codon frequency closer to that found naturally in highly expressed E. coli and human genes.

The compositions of the present invention can be delivered by a number of routes such as intramuscularly, subcutaneously, intraperitonally or intravenously.

In a preferred embodiment, the composition is delivered intradermally. In particular, the composition is delivered by means of a gene gun (particularly particle bombardment) administration techniques which involve coating the vector on to a bead (eg gold) which are then administered under high pressure into the epidermis; such as, for example, as described in Haynes et al, J Biotechnology 44: 37-42 (1996).

In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide, are accelerated to high speed within a helium gas jet generated by a hand held device, propelling the particles into a target tissue of interest, typically the skin. The particles are preferably gold beads of a 0.4-4.0 μm, more preferably 0.6-2.0 μm diameter and the DNA conjugate coated onto these and then encased in a cartridge or cassette for placing into the “gene gun”.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

It is possible for the immunogen component comprising the nucleotide sequence encoding the antigenic peptide, to be administered on a once off basis or to be administered repeatedly, for example, between 1 and 7 times, preferably between 1 and 4 times, at intervals between about 1 day and about 18 months. However, this treatment regime will be significantly varied depending upon the size of the patient, the disease which is being treated/protected against, the amount of nucleotide sequence administered, the route of administration, and other factors which would be apparent to a skilled medical practitioner.

The vectors which comprise the nucleotide sequences encoding antigenic peptides are administered in such amount as will be prophylactically or therapeutically effective. The quantity to be administered, is generally in the range of one picogram to 1 milligram, preferably 1 picogram to 10 micrograms for particle-mediated delivery, and 10 micrograms to 1 milligram for other routes of nucleotide per dose. The exact quantity may vary considerably depending on the weight of the patient being immunised and the route of administration.

Suitable techniques for introducing the naked polynucleotide or vector into a patient also include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical stimulation, electroporation and microseeding which is described in U.S. Pat. No. 5,697,901.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered.

In another embodiment the patient receives the antigen in different forms in a “prime boost” regime. Thus for example the antigen is first administered as adjuvanted protein formulation and then subsequently administered as a DNA based vaccine. This administration mode is preferred.

In another embodiment, the DNA based vaccine will be administered first, followed by the adjuvanted protein vaccine. Still another embodiment will concern the delivery of the DNA construct by means of specialised delivery vectors, preferably by the means of viral system, most preferably by the means of adenoviral-based systems.

Other suitable viral-based systems of DNA delivery include retroviral, lentiviral, adeno-associated viral, herpes viral and vaccinia-viral based systems. In another embodiment, the DNA based vaccine and the adjuvanted protein vaccine are co-administered to adjacent or overlapping sites.

A DNA sequence encoding the proteins of the present invention can be synthesized using standard DNA synthesis techniques, such as by enzymatic ligation as described by D. M. Roberts et al. in Biochemistry 1985, 24, 5090-5098, by chemical synthesis, by in vitro enzymatic polymerization, or by PCR technology utilising for example a heat stable polymerase, or by a combination of these techniques. Enzymatic polymerisation of DNA may be carried out in vitro using a DNA polymerase such as DNA polymerase I (Klenow fragment) in an appropriate buffer containing the nucleoside triphosphates dATP, dCTP, dGTP and dTTP as required at a temperature of 10″-37° C., generally in a volume of 50 μl or less. Enzymatic ligation of DNA fragments may be carried out using a DNA ligase such as T4 DNA ligase in an appropriate buffer, such as 0.05M Tris (pH 7.4), 0.01 M MgCl₂, 0.01 M dithiothreitol, 1 mM spermidine, 1 mM ATP and 0.1 mg/ml bovine serum albumin, at a temperature of 4° C. to ambient, generally in a volume of 50 ml or less. The chemical synthesis of the DNA polymer or fragments may be carried out by conventional phosphotriester, phosphite or phosphoramidite chemistry, using solid phase techniques such as those described in ‘Chemical and Enzymatic Synthesis of Gene Fragments—A Laboratory Manual’ (ed. H. G. Gassen and A. Lang), Verlag Chemie, Weinheim (1982), or in other scientific publications, for example M. J. Gait, H. W. D. Matthes, M. Singh, B. S. Sproat, and R. C. Titmas, Nucleic Acids Research, 1982, 10, 6243; B. S. Sproat, and W. Bannwarth, Tetrahedron Letters, 1983, 24, 5771; M. D. Matteucci and M. H. Caruthers, Tetrahedron Letters, 1980, 21, 719; M. D. Matteucci and M. H. Caruthers, Journal of the American Chemical Society, 1981, 103, 3185; S. P. Adams et al., Journal of the American Chemical Society, 1983, 105, 661; N. D. Sinha, J. Biernat, J. McMannus, and H. Koester, Nucleic Acids Research, 1984, 12, 4539; and H. W. D. Matthes et al., EMBO Journal, 1984, 3, 801.

In a further embodiment of the invention is provided a method of producing a protein as described herein. The process of the invention may be performed by conventional recombinant techniques such as described in Maniatis et al., Molecular Cloning—A Laboratory Manual; Cold Spring Harbor, 1982-1989. Accordingly there is provided a process for producing a xenogeneic polypeptide according to the invention, comprising culturing a host cell under conditions sufficient for the production of said polypeptide and recovering the polypeptide from the culture medium. In particular, the process of the invention may preferably comprise the steps of:
i) preparing a replicable or integrating expression vector capable, in a host cell, of expressing a DNA polymer comprising a nucleotide sequence that encodes the protein or an immunogenic derivative thereof;
ii) transforming a host cell with said vector;
ii) culturing said transformed host cell under conditions permitting expression of said DNA polymer to produce said protein; and
iv) recovering said protein.

The term ‘transforming’ is used herein to mean the introduction of foreign DNA into a host cell. This can be achieved for example by transformation, transfection or infection with an appropriate plasmid or viral vector using e.g. conventional techniques as described in Genetic Engineering; Eds. S. M. Kingsman and A. J. Kingsman; Blackwell Scientific Publications; Oxford, England, 1988. The term ‘transformed’ or ‘transformant’ will hereafter apply to the resulting host cell containing and expressing the foreign gene of interest. Preferably recombinant antigens of the invention are expressed in unicellular hosts, most preferably in bacterial systems, most preferably in E. coli.

The expression vectors are novel and also form part of the invention. The replicable expression vectors may be prepared in accordance with the invention, by cleaving a vector compatible with the host cell to provide a linear DNA segment having an intact replicon, and combining said linear segment with one or more DNA molecules which, together with said linear segment encode the desired product, such as the DNA polymer encoding the protein of the invention, or derivative thereof, under ligating conditions.

Thus, the hybrid DNA may be pre-formed or formed during the construction of the vector, as desired.

The choice of vector will be determined in part by the host cell, which may be prokaryotic or eukaryotic but are preferably E. coli, yeast or CHO cells. Suitable vectors include plasmids, bacteriophages, cosmids and recombinant viruses. Expression and cloning vectors preferably contain a selectable marker such that only the host cells expressing the marker will survive under selective conditions. Selection genes include but are not limited to the one encoding protein that confer a resistance to ampicillin, tetracyclin or kanamycin. Expression vectors also contain control sequences which are compatible with the designated host. For example, expression control sequences for E. coli, and more generally for prokaryotes, include promoters and ribosome binding sites. Promoter sequences may be naturally occurring, such as the -lactamase (penicillinase) (Weissman 1981, In Interferon 3 (ed. L. Gresser), lactose (lac) (Chang et al. Nature, 1977, 198: 1056) and tryptophan (trp) (Goeddel et al. Nucl. Acids Res. 1980, 8, 4057) and lambda-derived P_{L} promoter system. In addition, synthetic promoters which do not occur in nature also function as bacterial promoters. This is the case for example for the tac synthetic hybrid promoter which is derived from sequences of the trp and lac promoters (De Boer et al., Proc. Natl Acad. Sci. USA 1983, 80, 21-26). These systems are particularly suitable with E. coli.

Yeast compatible vectors also carry markers that allow the selection of successful transformants by conferring prototrophy to auxotrophic mutants or resistance to heavy metals on wild-type strains. Control sequences for yeast vectors include promoters for glycolytic enzymes (Hess et al., J. Adv. Enzyme Reg. 1968, 7, 149), PHO5 gene encoding acid phosphatase, CUP1 gene, ARG3 gene, GAL genes promoters and synthetic promoter sequences. Other control elements useful in yeast expression are terminators and leader sequences. The leader sequence is particularly useful since it typically encodes a signal peptide comprised of hydrophobic amino acids which direct the secretion of the protein from the cell. Suitable signal sequences can be encoded by genes for secreted yeast proteins such as the yeast invertase gene and the a-factor gene, acid phosphatase, killer toxin, the a-mating factor gene and recently the heterologous inulinase signal sequence derived from INU1A gene of Kluyveromyces marxianus. Suitable vectors have been developed for expression in Pichia pastoris and Saccharomyces cerevisiae.

A variety of P. pastoris expression vectors are available based on various inducible or constitutive promoters (Cereghino and Cregg, FEMS Microbiol. Rev. 2000, 24:45-66). For the production of cytosolic and secreted proteins, the most commonly used P. pastoris vectors contain the very strong and tightly regulated alcohol oxidase (AOX1) promoter. The vectors also contain the P. pastoris histidinol dehydrogenase (HIS4) gene for selection in his4 hosts. Secretion of foreign protein require the presence of a signal sequence and the S. cerevisiae prepro alpha mating factor leader sequence has been widly and successfully used in Pichia expression system. Expression vectors are integrated into the P. pastoris genome to maximize the stability of expression strains. As in S. cerevisiae, cleavage of a P. pastoris expression vector within a sequence shared by the host genome (AOX1 or HIS4) stimulates homologous recombination events that efficiently target integration of the vector to that genomic locus. In general, a recombinant strain that contains multiple integrated copies of an expression cassette can yield more heterologous protein than single-copy strain. The most effective way to obtain high copy number transformants requires the transformation of Pichia recipient strain by the sphaeroplast technique (Cregg et all 1985, Mol. Cell. Biol. 5: 3376-3385).

The preparation of the replicable expression vector may be carried out conventionally with appropriate enzymes for restriction, polymerisation and ligation of the DNA, by procedures described in, for example, Maniatis et al. cited above.

The recombinant host cell is prepared, in accordance with the invention, by transforming a host cell with a replicable expression vector of the invention under transforming conditions. Suitable transforming conditions are conventional and are described in, for example, Maniatis et al. cited above, or “DNA Cloning” Vol. II, D. M. Glover ed., IRL Press Ltd, 1985. The choice of transforming conditions depends upon the choice of the host cell to be transformed. For example, in vivo transformation using a live viral vector as the transforming agent for the polynucleotides of the invention is described above. Bacterial transformation of a host such as E. coli may be done by direct uptake of the polynucleotides (which may be expression vectors containing the desired sequence) after the host has been treated with a solution of CaCl₂ (Cohen et al., Proc. Nat. Acad. Sci., 1973, 69, 2110) or with a solution comprising a mixture of rubidium chloride (RbC1), MnCl₂, potassium acetate and glycerol, and then with 3-[N-morpholino]-propane-sulphonic acid, RbC1 and glycerol. Transformation of lower eukaryotic organisms such as yeast cells in culture by direct uptake may be carried out by using the method of Hinnen et al (Proc. Natl. Acad. Sci. 1978, 75: 1929-1933). Mammalian cells in culture may be transformed using the calcium phosphate co-precipitation of the vector DNA onto the cells (Graham & Van der Eb, Virology 1978, 52, 546). Other methods for introduction of polynucleotides into mammalian cells include dextran mediated transfection, polybrene mediated transfection, protoplast fusion, electroporation, encapsulation of the polynucleotide(s) into liposomes, and direct micro-injection of the polynucleotides into nuclei.

The invention also extends to a host cell transformed with a nucleic acid encoding the protein of the invention or a replicable expression vector of the invention. Culturing the transformed host cell under conditions permitting expression of the DNA polymer is carried out conventionally, as described in, for example, Maniatis et al. and “DNA Cloning” cited above. Thus, preferably the cell is supplied with nutrient and cultured at a temperature below 50° C., preferably between 25° C. and 35° C., most preferably at 30° C. The incubation time may vary from a few minutes to a few hours, according to the proportion of the polypeptide in the bacterial cell, as assessed by SDS-PAGE or Western blot.

The product may be recovered by conventional methods according to the host cell and according to the localisation of the expression product (intracellular or secreted into the culture medium or into the cell periplasm). Thus, where the host cell is bacterial, such as E. coli it may, for example, be lysed physically, chemically or enzymatically and the protein product isolated from the resulting lysate. Where the host cell is mammalian, the product may generally be isolated from the nutrient medium or from cell free extracts. Where the host cell is a yeast such as Saccharomyces cerevisiae or Pichia pastoris, the product may generally be isolated from from lysed cells or from the culture medium, and then further purified using conventional techniques. The specificity of the expression system may be assessed by western blot using an antibody directed against the polypeptide of interest.

Conventional protein isolation techniques include selective precipitation, adsorption chromatography, and affinity chromatography including a monoclonal antibody affinity column. When the proteins of the present invention are expressed with a histidine tail (His tag), they can easily be purified by affinity chromatography using an ion metal affinity chromatography column (IMAC) column.

In a preferred embodiment of the invention the proteins of the present invention is provided with an affinity tag, such as a polyhistidine tail. In such cases the protein after the blocking step is preferably subjected to affinity chromatography. For those proteins with a polyhistidine tail, immobilised metal ion affinity chromatography (IMAC) may be performed. The metal ion, may be any suitable ion for example zinc, nickel, iron, magnesium or copper, but is preferably zinc or nickel. Preferably the IMAC buffer contains detergent, preferably a non-ionic detergent such as Tween 80, or a zwitterionic detergent such as Empigen BB, as this may result in lower levels of endotoxin in the final product.

Further chromatographic steps include for example a Q-Sepharose step that may be operated either before of after the IMAC column. Preferably the pH is in the range of 7.5 to 10, more preferably from 7.5 to 9.5, optimally between 8 and 9, ideally 8.5.

The proteins of the present invention are provided either soluble in a liquid form or in a lyophilised form, which is the preferred form. It is generally expected that each human dose will comprise 1 to 1000 μg of protein, and preferably 30-300 μg.

The present invention also provides pharmaceutical/immunogenic and vaccine composition comprising xenogeneic P501S antigen or nucleic acid in a pharmaceutically acceptable excipient. Accordingly there is provided a process for the production of an immunogenic composition, comprising admixing a xenogeneic P501S polypeptide or a xenogeneic P501S-encoding polynucleotide with a suitable adjuvant, diluent or other pharmaceutically acceptable carrier.

More particularly the pharmaceutical/immunogenic and vaccine compositions of the invention comprise an effective amount of a xenogeneic P501S polypeptide or a xenogeneic P501S-encoding polynucleotide, and a pharmaceutically acceptable carrier. By effective amount is meant a dose of antigen that, when administered to a human, produces a detectable immune response, such as a humoral response (antibodies) or a cellular response. A preferred immunogenic composition comprises at least one xenogeneic P501S polypeptide having the sequence set forth in SEQ ID NO:1, in SEQ ID NO:3 or in SEQ ID NO:10 or an immunogenic fragment thereof.

Said protein has, preferably, blocked thiol groups and is highly purified, e.g. has less than 5% host cell contamination. Another preferred immunogenic composition comprises at least one xenogeneic P501S-encoding polynucleotide having the sequence set forth in SEQ ID NO:2, in SEQ ID NO:4 or in SEQ ID NO:11 or a fragment thereof which encodes a polypeptide having retained some functional similarity with the protein of SEQ ID NO:1, in SEQ ID NO:3 or in SEQ ID NO:10. Such vaccine may optionally contain one or more other tumour-associated antigen and derivatives from human or non-human origin. For example, suitable other associated antigen include PAP-1, PSA (prostate specific antigen), PSMA (prostate-specific membrane antigen), PSCA (Prostate Stem Cell Antigen), STEAP.

Vaccine preparation is generally described in Vaccine Design (“The subunit and adjuvant approach” (eds. Powell M. F. & Newman M. J). (1995) Plenum Press New York). Encapsulation within liposomes is described by Fullerton, U.S. Pat. No. 4,235,877.

The xenogenic proteins are preferably adjuvanted in the pharmaceutical/immunogenic or vaccine formulation of the invention. Suitable adjuvants are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); SBAS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

In the formulations of the invention it is preferred that the adjuvant composition induces an immune response predominantly of the TH1 type. High levels of Th1-type cytokines (e.g., IFN-γ, TNFα, IL-2 and IL-12) tend to favour the induction of cell mediated immune responses to an administered antigen. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Accordingly, suitable adjuvants for use in eliciting a predominantly Th1-type response include, for example a combination of monophosphoryl lipid A, preferably 3-de-O-acylated monophosphoryl lipid A (3D-MPL) together with an aluminium salt. Other known adjuvants which preferentially induce a TH1 type immune response include CpG containing oligonucleotides. The oligonucleotides are characterised in that the CpG dinucleotide is unmethylated. Such oligonucleotides are well known and are described in, for example WO 96/02555. Immunostimulatory DNA sequences are also described, for example, by Sato et al., Science 273:352, 1996. Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

A particularly potent adjuvant formulation involving QS21 3D-MPL & tocopherol in an oil in water emulsion is described in WO 95/17210 and is a preferred formulation.

Other preferred adjuvants include Montanide ISA 720 (Seppic, France), SAF (Chiron, Calif., United States), ISCOMS (CSL), MF-59 (Chiron), Detox (Ribi, Hamilton, Mont.), RC-529 (Corixa, Hamilton, Mont.) and other aminoalkyl glucosaminide 4-phosphates (AGPs).

Other preferred adjuvants include adjuvant molecules of the general formula (I):
HO(CH₂CH₂O)ₙ-A-R
Wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549. The polyoxyethylene ether according to the general formula (I) above may, if desired, be combined with another adjuvant, preferably with CpG.

Accordingly in one embodiment of the present invention there is provided a vaccine comprising a xenogeneic P501S of the present invention, which additionally comprises a TH-1 inducing adjuvant. A preferred embodiment is a vaccine in which the TH-1 inducing adjuvant is selected from the group of adjuvants comprising: 3D-MPL, QS21, a mixture of QS21 and cholesterol, and a CpG oligonucleotide. Another preferred embodiment is a vaccine comprising a xenogeneic P501S adjuvanted with a monophosphoryl lipid A or derivative thereof, QS21 and tocopherol in an oil in water emulsion.

Preferably the vaccine additionally comprises a saponin, more preferably QS21. Another particular suitable adjuvant formulation including CpG and a saponin is described in WO 00/09159 and is a preferred formulation. Most preferably the saponin in that particular formulation is QS21. Preferably the formulation additionally comprises an oil in water emulsion and tocopherol.

Any of a variety of delivery vehicles may be employed within pharmaceutical compositions and vaccines to facilitate production of an antigen-specific immune response that targets tumour cells. Delivery vehicles include antigen-presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumour effects per se and/or to be immunologically compatible with the receiver (i.e., matched HLA haplotype). APCs may generally be isolated from any of a variety of biological fluids and organs, including tumour and peri-tumoural tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumour immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naïve T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998). Accordingly there is preferably provided a vaccine comprising an effective amount of dendritic cells or antigen presenting cells, modified by in vitro loading with a polypeptide as described herein, or genetically modified in vitro to express a polypeptide as described herein and a pharmaceutically effective carrier.

Dendritic cells and progenitors may be obtained from peripheral blood, bone marrow, tumour-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, lipopolysaccharide LPS, flt3 ligand and/or other compound(s) that induce differentiation, maturation and proliferation of dendritic cells. Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding P501S tumour protein (or derivative thereof) such that the P501S tumour polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the P501S tumour polypeptide, DNA (naked or within a plasmid vector) or RNA; or with antigen-expressing recombinant bacterium or viruses (e.g., vaccinia, fowlpox, adenovirus or lentivirus vectors).

Vaccines and pharmaceutical/immunogenic compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

The present invention also provides a method of inducing an immune response against human P501S having an amino acid sequence as set forth in any of the sequences SEQ ID NO:5 to SEQ ID NO:7 in a human, comprising administering to the subject an effective dosage of a composition comprising a xenogeneic form of said human P501S as described herein. A preferred embodiment is a method of inducing an immune response against human P501S using the xenogeneic P501S isolated from mouse, rat or Cynomolgus monkey. Another preferred method of inducing an immune response according to the present invention is using an antigen composition including a live viral expression system which expresses said xenogeneic antigen said.

The present invention also provides a method for producing a vaccine formulation comprising mixing a protein of the present invention together with a pharmaceutically acceptable excipient, such as 3D-MPL.

Another aspect of the invention is the use of a polypeptide or a polynucleotide as claimed herein in the manufacture of a pharmaceutical/immunogenic or vaccine for immunotherapeutically treating a patient suffering from or susceptible to prostate cancer or other P501S-associated tumours or diseases.

### FIGURE LEGENDS

FIG. 1: amino acid sequence for rat P501S (SEQ ID No 1).

FIG. 2: nucleotide sequence encoding rat P501S (SEQ ID No 2). The ORF appears in lower case.

FIG. 3: amino acid sequence for Cynomolgus monkey P501S (SEQ ID No 3).

FIG. 4: nucleotide sequence encoding Cynomolgus monkey P501S (SEQ ID No 4). The ORF appears in lower case.

FIG. 5: amino acid sequence for human P501S (SEQ ID No 5).

FIG. 6: amino acid sequence for human P501S (SEQ ID No 6).

FIG. 7: amino acid sequence for human P501S (SEQ ID No 7).

FIG. 8: design of the alpha prepro P501S His protein expressed in Saccharomyces cerevisiae.

FIG. 9: amino acid sequence (SEQ ID NO:9) and nucleotide sequences (SEQ ID NO:8) of alpha prepro P501S his tailed recombinant protein expressed in Saccharomyces cerevisiae

FIG. 10: Saccharomyces cerevisiae (strain Y1790) P501S-His fermentation process

FIG. 11: ELISPOT responses following fours immunisations with pVAC empty and pVAC-P501S (JNW680). Male C57BL/6 mice

FIG. 12: ELISPOT responses following four immunisations with pVAC empty and pVAC-P501S (JNW680). Female C57BL/6 mice

FIG. 13: Real-time PCR analysis of P501S on Cynomolgus prostate and on a panel of rat tissues and cell lines. Abbreviations are depicted in Table 2.

FIG. 14: amino acid sequence for mouse P501S (SEQ ID No 10)

FIG. 15: nucleotide sequence for mouse P501S (SEQ ID No 11). The ORF appears in lower case.

FIG. 16: Real-time PCR analysis of P501S on a panel of mouse tissues.

FIG. 17: Expression of mouse and human P501S

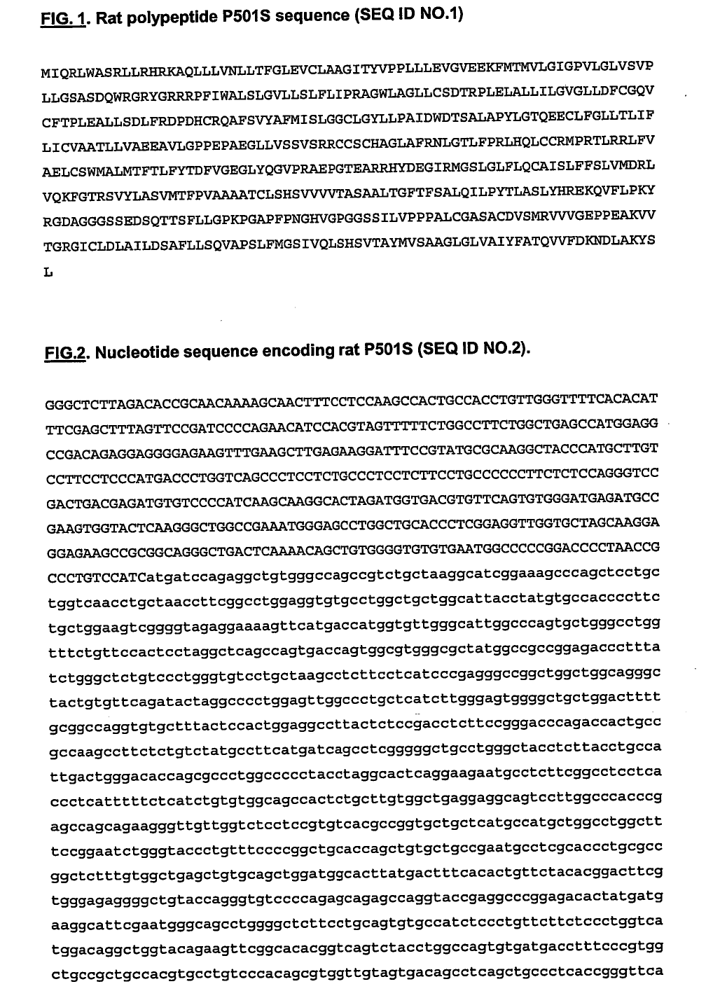

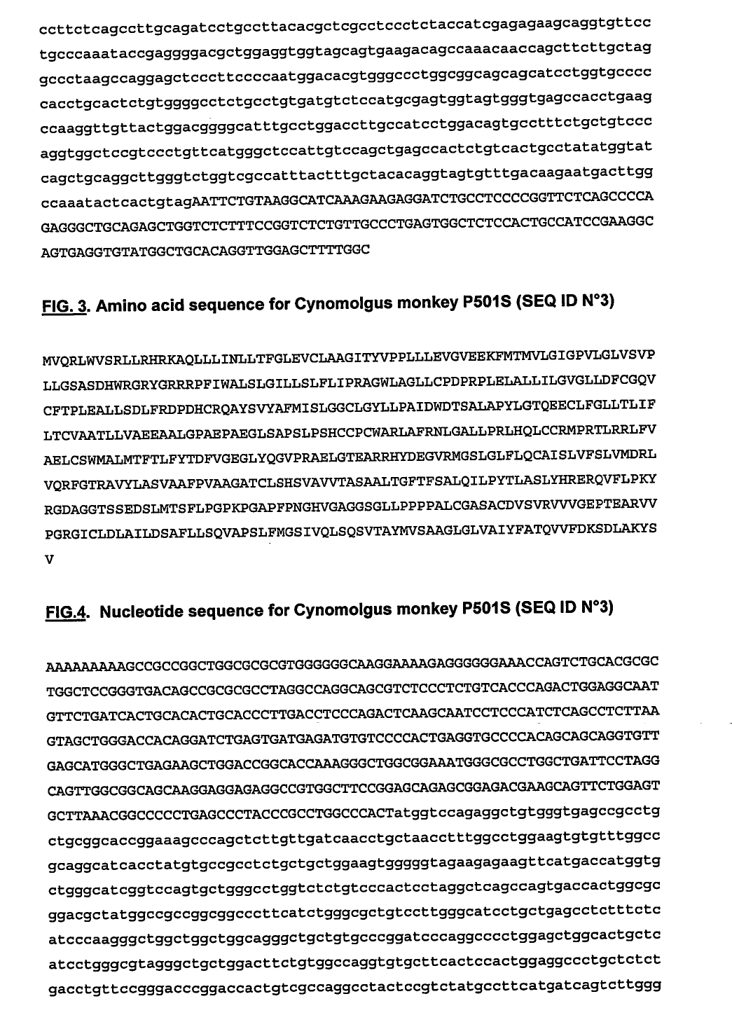

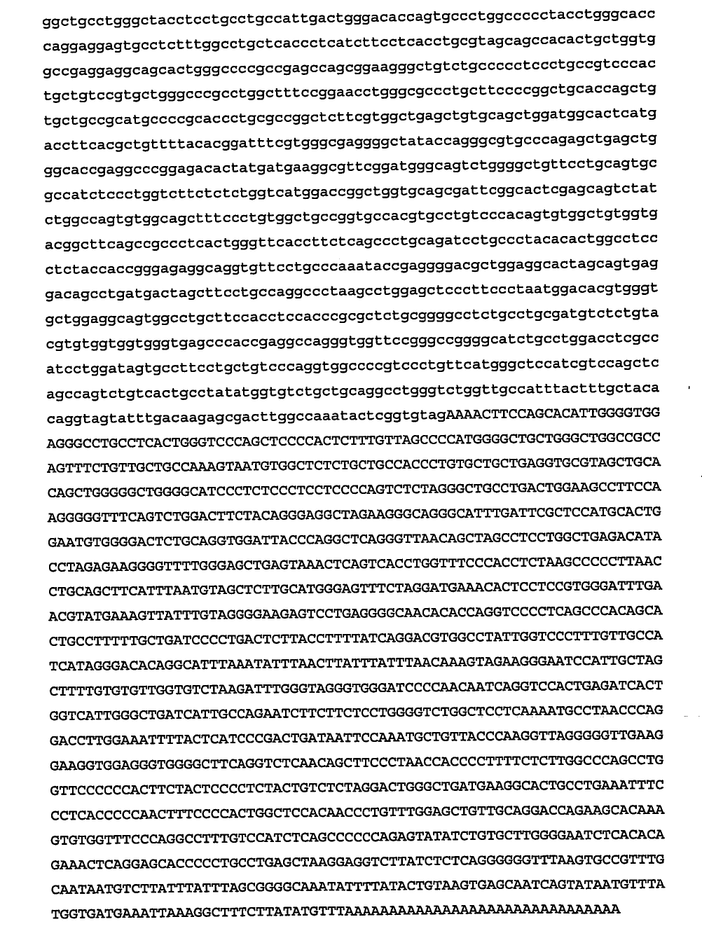

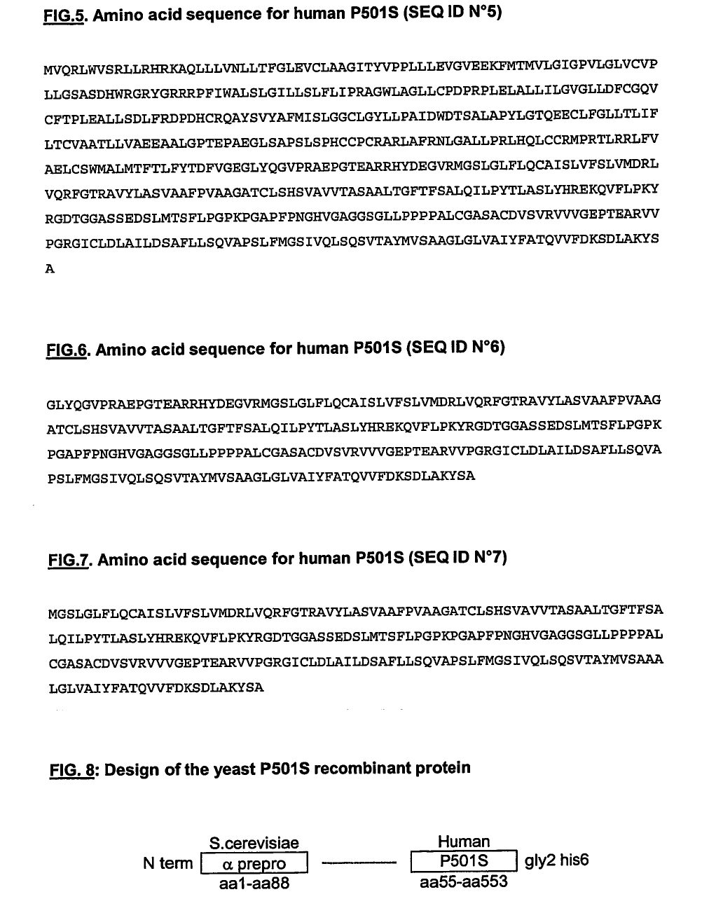

The invention will be further described by reference to the following examples:

### EXAMPLE I

Preparation of Recombinant Yeast Strain Saccharomyces cerevisiae Expressing Alphaprepro P501S His Tailed, Under Cup1 Promoter

1.—Introduction

The yeast expression system detailed below is suitable to express:

i) either recombinant non-human (monkey, rat, mouse for example) protein to be formulated subsequently in a vaccine or pharmaceutical/immunogenic composition to be inoculated into humans. Xenogeneic P501S can be expressed with its own signal sequence or with alpha prepro signal sequence (similarly to what is illustrated below).

ii) or recombinant human P501S protein to be formulated subsequently in a vaccine or pharmaceutical/immunogenic composition to be inoculated into animals (monkeys, rabbits, mouse or rat for example).

The Example below describes the expression of human P501S in yeast.

In order to target P501S protein in yeast endoplasmic reticulum (ER) membrane, the native secretion signal sequence and putative first lumenal domain was replaced by yeast alpha prepro signal sequence, in such a way that the natural position in membrane was conserved. The preparation of recombinant strain Saccharomyces cerevisiae Y1790 expressing P501S as well as characterization of recombinant protein are described below.

2.—Protein Design

The native secretion signal sequence and first putative lumenal domain of P501S protein was replaced by Saccharomyces cerevisiae alpha prepro signal sequence. The yeast signal sequence was fused to the N terminus of P501S sequence, coding from amino acid 55 to amino acid 553 (end of protein). The C terminal end of the recombinant protein was elongated by 2 glycines and six histidines (FIG. 8).

3.—Construction of pRIT15068 Plasmid for Saccharomyces cerevisae Expression

The starting material was the recombinant plasmid P501S, derived from commercial plasmid pcDNA3.1 (Invitrogen) containing a 3,4 Kb insert between EcoRI and NotI cloning restriction sites. This plasmid contains the P501S full length coding sequence (1662 bp long) and was obtained from Corixa Corporation. The cloning strategy includes the following steps:

a. Subcloning of P501S:

A 1569 bp fragment containing nucleotide sequence coding for last 499 aminoacids+68 bp in aval of the P501S open reading frame was isolated from Corixa p501S plasmid by Nco I digest. After T4 polymerase treatment, the fragment was subcloned in plasmid pUC18 opened by PstI and XbaI, T4 polymerase treated, in such a way that NcoI was recovered within the N terminal sequence of P501S open reading frame (i.e. amino acid position 55). The plasmid obtained was called pRIT15061.

b. Introduction of S. cerevisiae CUP1 Promoter and Yeast Alpha Prepro Signal Sequence:

A PCR fragment containing the yeast CUP1 promoter and yeast alpha prepro signal sequence was obtained by 3 successive PCR steps:

PCR step 1: the amplification of CUP1 promoter with oligonucleotides MDENHE1CUP1 (c 5′ GGA CTA GTC TAG CTA GCT TGC TGT CAG TCA CTG TCA AGA G 3′) and MDECUP1ATG (nc 5′CAT TTT ATG TGA TGA TTG ATT G 3′) was performed on pRIT12471 plasmid as template.

pRIT12471 was obtained as follows: plasmid Yep6-36 harbouring the CUP-1 gene (Butt T R et al., Proc Natl Acad Sci USA. 1984 June;81(11):3332-6) was received from TR. Butt (SmithKline Beecham Pharmaceuticals, Research and Development, King of Prussia, Pa., USA). A BamHI-BbvI fragment (468 base pairs) containing the CUP-1 promoter and the N-terminal coding sequences was isolated from Yep6-36 plasmid, and treated with Bal31 enzyme in order to remove the N-terminal coding region and place a BamHI site adjacent to the ATG. After Bal31 treatment the DNA fragments were inserted into pAB119, a pBR322 like plasmid previously digested by BamHI and T4 polymerase repared. Several derivative plasmids were obtained and sequenced, amongst which pRIT12471.

PCR step 2: the amplification of alpha preprosignal sequence with oligonucleotides MDEPREPROAT (c 5′CAA TCA ATC MT CAT CAC ATA MA TGA GAT TTC CTT CM TTT TTA CTG CA 3′) and MDESIGNAL2 (nc5′ GCT AGC TCC ATG GCT TCA GCC TCT CTT TTC TCG AG 3′) was performed on pPIC9 plasmid (INVITROGEN) as template.

PCR step 3: the association of CUP1 promoter and alpha preprosignal sequence by PCR was performed using the fragments obtained by PCR step 1 and PCR step 2 and oligonucleotides MDENHE1CUP1 and MDESIGNAL2. After this step, the amplified fragment was purified, treated with T4 polymerase and digested by NcoI. The resulting fragment was introduced into plasmid pRIT15061 between the HindIII site treated with T4 polymerase, and the NcoI site. This resulting plasmid was called pRIT15062.

C. Elongation of the C Terminus by HIS Tail:

The fragment for HIS tail elongation was obtained by PCR using p501S plasmid as a template and oligonucleotides MDE501SAC (c 5′CTG GAG GTG CTA GCA GTG AG 3′) and MDE501HIS (nc 5′CTA GTC TAG AGA ATT CCC CGG GTT MT GGT GAT GGT GAT GGT GTC CAC CCG CTG AGT ATT TGG CCA AGT CG 3′). The amplified fragment was purified and digested by SacI and EcoRI and introduced between SacI (overlapping amino acid 43) and EcoRI sites in pRIT 15062 plasmid, restauring correct open reading frame and elongating, in frame, p501S sequence by sequence coding for 2 glycines, 6 histidines, a stop codon. Moreover a SmaI site and EcoRI site are still introduced. This plasmid was called pRIT15063.

d. Introduction of Promoter and Coding Sequence in Yeast Expression Vector

The FspI-SmaI fragment carrying the promoter and the recombinant P501S coding sequence was isolated from pRIT15063 plasmid and cloned in BamHI site, treated with T4 polymerase, of pRIT 15073 plasmid in such a way that the fragment was oriented with the C terminal of protein near the ARG3 terminator sequence. This last plasmid is a E. coli S. cerevisiae shuttle vector carrying LEU2 gene for yeast complementation and the complete 2 micron sequence. This ligation leads to pRIT15067 plasmid.

e. An Unexpected Nucleotide Deletion was Found Out in Alpha Prepro Sequence, so the Last Step was Performed to Restore the Sequence:

The full-length p501S coding sequence and the vector sequence were recovered from pRIT15067 plasmid on 2 fragments NcoI/SalI and SalI/NheI. A new fragment carrying CUP1 promoter and yeast alpha prepro signal sequence was isolated as described in step b and digested by NheI and NcoI. These 3 fragments were ligated together to obtain pRIT15068 expression plasmid. In this plasmid, P501S expression is driven by yeast CUP1 promoter.

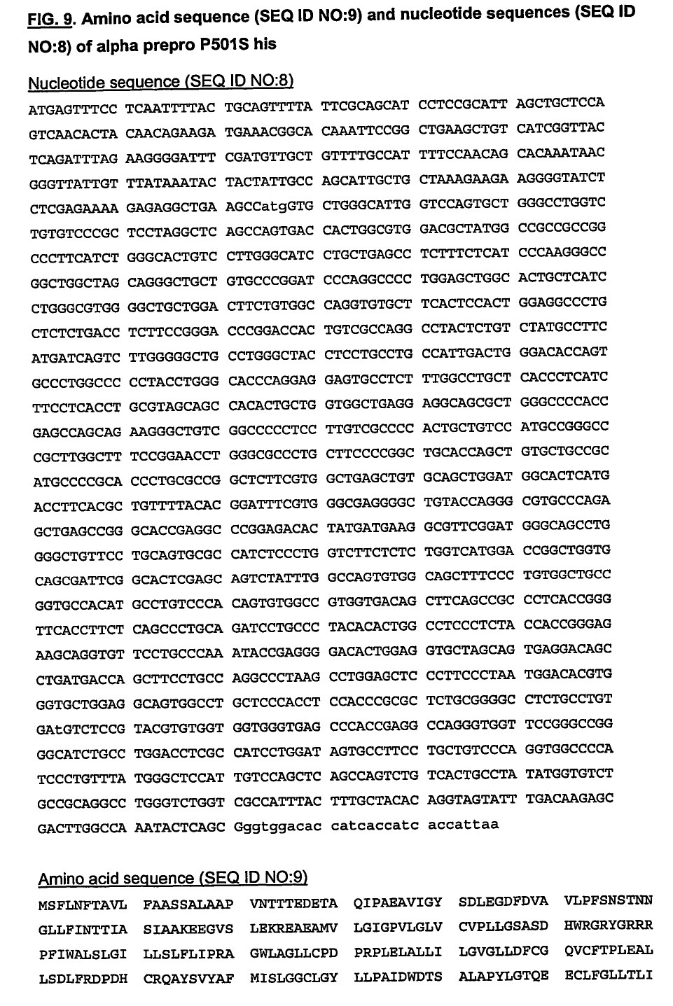

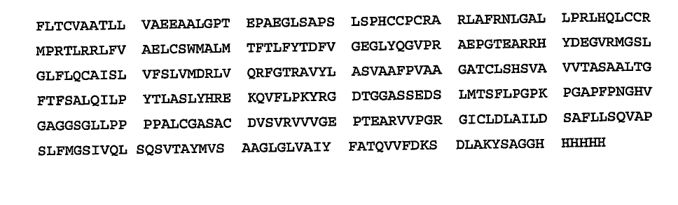

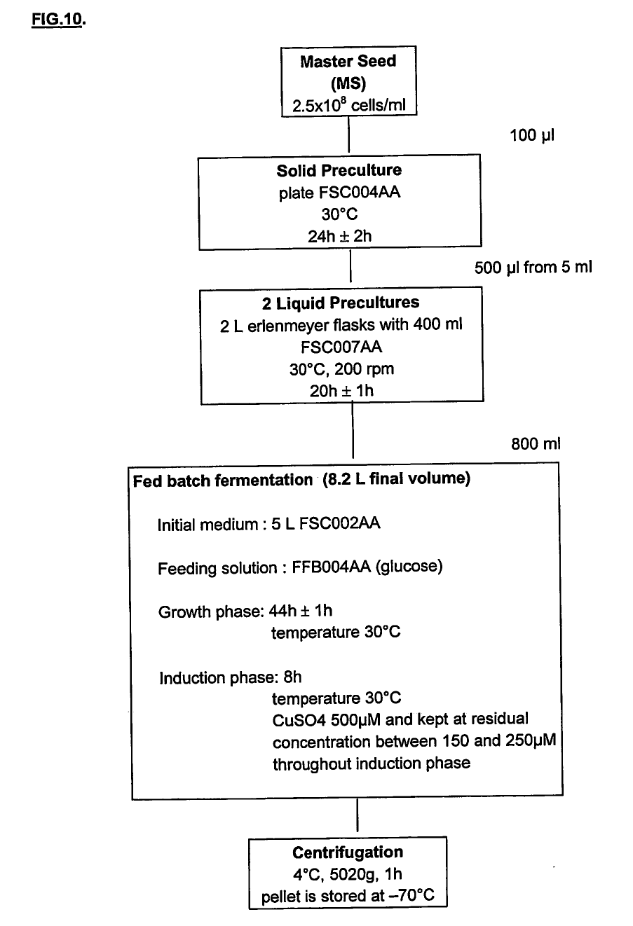

The nucleotide (SEQ ID NO:8) and amino acid sequence (SEQ ID NO:9) of the recombinant protein are illustrated in FIG. 9.

4.—Transformation of S. cerevisiae DC5 Strain and Generation of Y1790 Strain

The transformation of DC5 strain (a his3 leu 2-3 leu 2-112 can1-11) was performed by the lithium acetate method (Methods in enzymology, 1991, vol 194, pg 186) using plasmid pRIT15068. Yeast cells were spread on minimal medium plus histidine. Transformants were picked and tested for expression. Y1790 was one of these transformants.

5.—Induction of S. cerevisiae Strain Y1790

Strain Y1790 was grown, at 30° C., in minimal medium supplemented with glucose 2% and histidine 80 ng/ml. Yeast cells were harvested in exponential growing phase and resuspended to a final OD=0.5 in same medium supplemented with CuSO₄ to final concentration of 500 μg/ml for induction. Culture is maintained at 30° during 24 h and then, cells are harvested for expression analysis.

### EXAMPLE II

Expression and Characterization of Recombinant p501S Protein.

1.—Highlights

Using the process described below, the P501S antigen produced was clearly identified as a 62 KD major band by Western Blot analysis. The antigen productivity was compared by WB analysis and densitometry. The antigen was located in the insoluble fraction obtained from the cell homogenate after centrifugation. The specific antigen productivity of strain Y1790 in fermenters was approximately 4 times higher than in flasks. As the biomass was amplified by a factor 10 in fermenter, the volumetric productivity was about 40 times higher in fermenter compared to flask cultures. Strain Y1790 (his-) was grown in fed-batch fermentation using 20 L vessels.

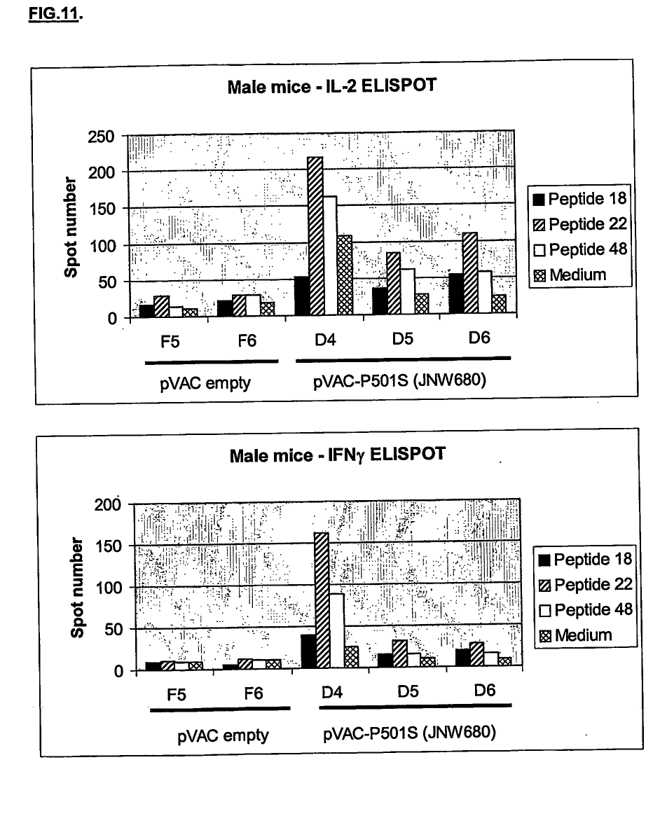

2.—Process Description for Strain Y1790 (FIG. 10)

a. Pre-Cultures

100 μl of this lab Master Seed (MS) containing 2.5×10⁸ cfu/ml were spread on FSC004AA solid medium (see medium composition below). Two plates were incubated for 26 h at 30° C. These solid pre-cultures were harvested in 5 ml of liquid medium FSC007AA each and 0.5 ml (or 9.3×10⁷ cells) of this suspension was used to inoculate each of the 2 liquid pre-cultures.

These pre-cultures were run for 20 hours in 2 L flasks containing 400 ml of medium FSC007AA in order to obtain an OD of 1.8. The other characteristics of these pre-cultures are the following: pH 2.8-glucose 2.3 g/L-ethanol 3.4 g/L.

The best timing for liquid pre-cultures for strain Y1790 was determined in preliminary experiments. Liquid pre-cultures containing 400 ml of medium and inoculated with various volumes of MS (0.25, 0.5, 1 and 2 ml) was monitored in order to identify the best inoculum size and timing for process. Glucose, ethanol, pH and OD and cell number (flow cytometry) were followed between 16 and 23 hours of culture. Glucose exhaustion and maximal biomass were obtained after 20 hour incubation with 0.5 inoculum. These conditions were adopted for transferring the pre-culture into fermentation.

a. Fermentation Process

In total, 800 ml of pre-culture were used to inoculate a 20 L fermenter containing 5 L of medium FSC002AA. 3 ml of irradiated antifoam were added before inoculation. The carbon source (glucose) was supplemented to the culture by a continuous feeding of the FFB004AA medium. The residual glucose concentration was maintained very low (≦50 mg/L) in order to minimise the ethanol production by fermentation. This was realised by limiting the development of the micro-organism by limited glucose feed rate. The Standard biomass content (OD 80-90) for DC5 host strain was reached in fermentation after 44 hour growth phase.

CUP1 promoter was then induced by adding CuSO4 500 μM in order to produce P501S antigen. CuSO4 addition was followed by ethanol accumulation (up to 6 g/L), and glucose feeding rate was then reduced in order to consume the ethanol produced. The copper available for the microorganism was monitored by testing Cu ion concentration in the broth supernatant using a spectrophotometric copper assay (DETC method).

The fermentation was then supplemented by CuSO4 throughout the induction phase in order to maintain its concentration between 150 and 250 μM in the supernatant. The biomass reached an OD of 100 at the end of induction. Culture was harvested after 8 hours of induction.

C. Antigen Characterisation and Productivity

The cell homogenate was prepared and analysed by SDS-PAGE and Western Blot (WB) using standard protocols. A major protein band with the expected MW of 62 KD was detected by WB using Corixa monoclonal P501S antibodies. WB analysis also showed that the major 62 KD band was progressively produced from 30 minutes of induction on, and reached a maximum after 3 hours. No more antigen seemed to be produced between 3 and 12 hours of induction. The number of passages through French Press necessary to extract all the antigen from the cells was evaluated. One, three and five passages were tested and total cell lysates, supernatants and pellets of cell lysates were analysed by WB. Three passages through French Press were sufficient to completely extract the antigen. Nothing was visible in the supernatants, the antigen was associated to the insoluble fraction. A washing step will facilitate the purification by elimination of a part of the soluble proteins.

d. Culture Media Composition

FFB004AA

Glucose:350 g/l; Na2MoO4.2H2O:5.15 mg/l; Acide folique: 1.36 mg/l; KH2PO4: 20.6 g/l; MnSO4.H2O:10.3 mg/l; Inositol: 1350 mg/l; MgSO4.7H2O:11.7 g/l; H₃BO3:12.9 m/l; Pyridoxine:170 mg/l; CaCl2.2H2O:2.35 g/l; KI:2.6 mg/l; Thiamine:170 g/l; NaCl:0.15 g/l; CoCl2.6H2O:2.3 mg/l; Niacine:0.67 mg/l; HCl:2.5 ml/l; FeCl3.6H2O:24.8 mg/l; Riboflavine:0.33 mg/l; CuSO4.5H2O:1.03 mg/l; Biotine:1.36 mg/l; Panthotenate Ca:170 mg/l; ZnSO4.7H2O:10.3 mg/l; Para-aminobenzoic acid: 0.33 mg/l; Histidine:5.35 g/l.

FSC007AA

Glucose:10 g/l; Na2MoO4.2H2O:0.0002 g/l; Acide folique:0.000064 g/l; KH2PO4:1 g/l; MnSO4.H2O:0.0004 g/l; Inositol:0.064 g/l; MgSO4.7H2O:0.5 g/l; H₃BO3:0.0005 g/l; Pyridoxine:0.008 g/l; CaCl2.2H2O:0.1 g/l; KI:0.0001 g/l; Thiamine:0.008 g/l; NaCl:0.1 g/l; CoCl2.6H2O:0.00009 g/l; Niacine:0.000032 g/l; FeCl3.6H2O:0.0002 g/l; Riboflavine:0.000016 g/l; Panthotenate Ca:0.008 g/l; CuSO4.5H2O:0.00004 g/l; Biotine:0.000064 g/l; para-aminobenzoic acid: 0.000016 g/l; ZnSO4.7H2O:0.0004 g/l; (NH4)₂SO4:5 g/l; Histidine:0.1 g/l.

FSC002AA

(NH4)2SO4:6.4 g/l; Na2MoO4.2H2O: 2.05 mg/l; Acide folique: 0.54 mg/l; KH2PO4:8.25 g/l; MnSO4.H2O:4.1 mg/l; Inositol:540 mg/l; MgSO4.7H2O:4.69 g/l; H₃BO3:5.17 m/l; Pyridoxine:68 mg/l; CaCl2.2H2O:0.92 g/l; KI:1.03 mg/l; Thiamine:68 mg/l; NaCl:0.06 g/l; CoCl2.6H2O:0.92 mg/l; Niacine:0.27 mg/l; HCl:1 ml/l; FeCl3.6H2O:9.92 mg/l; Riboflavine:0.13 mg/l; CuSO4.5H2O:0.41 mg/l; Glucose:0.14 g/l; Panthotenate Ca:68 mg/l; ZnSO4.7H2O:4.1 mg/l; Biotine:0.54 mg/l; para-aminobenzoic acid: 0.13 mg/l; Histidine:0,3 g/l.

FSC004AA

Glucose:10 g/l; Na2MoO4.2H2O:0.0002 g/l; Acide folique: 0.000064 g/l; KH2PO4: 1 g/l; MnSO4.H2O:0.0004 g/l; Inositol:0.064 g/l; MgSO4.7H2O:0.5 g/l; H₃BO3:0.0005 g/l; Pyridoxine:0.008 g/l; CaCl2.2H2O:0.1 g/l; KI:0.0001 g/l; Thiamine:0.008 g/l; NaCl: 0.1 g/l; CoCl2.6H2O:0.00009 g/l; Niacine:0.000032 g/l; FeCl3.6H2O:0.0002 g/l; Riboflavine:0.000016 g/l; Panthotenate Ca:0.008 g/l; CuSO4.5H2O:0.00004 g/l; Biotine:0.000064 g/l; para-aminobenzoic acid:0.000016 g/l; ZnSO4.7H2O:0.0004 g/l; (NH4)2SO4: 5 g/l; Agar 18 g/l; Histidine:0.1 g/l.

### EXAMPLE III

Compositions and Methods to Induce an Immune Response

A—Vaccine Preparation Using Xenogeneic or Human P501S

1.—Vaccine Preparation:

The vaccine used in these experiments is produced from a recombinant DNA, encoding a human or xenogeneic P501S recombinantly expressed in S. cerevisiae, either adjuvanted or not. As an adjuvant, the formulation comprises a mixture of 3 de-O-acylated monophosphoryl lipid A (3D-MPL) and QS21 in an oil/water emulsion. The adjuvant system SBAS2 has been previously described WO 95/17210.

3D-MPL: is an immunostimulant derived from the lipopolysaccharide (LPS) of the Gram-negative bacterium Salmonella minnesota. MPL has been deacylated and is lacking a phosphate group on the lipid A moiety. This chemical treatment dramatically reduces toxicity while preserving the immunostimulant properties (Ribi, 1986). Ribi Immunochemistry produces and supplies MPL to SB-Biologicals. Experiments performed at Smith Kline Beecham Biologicals have shown that 3D-MPL combined with various vehicles strongly enhances both the humoral and a TH1 type of cellular immunity.

QS21: is a natural saponin molecule extracted from the bark of the South American tree Quillaja saponaria Molina. A purification technique developed to separate the individual saponines from the crude extracts of the bark, permitted the isolation of the particular saponin, QS21, which is a triterpene glycoside demonstrating stronger adjuvant activity and lower toxicity as compared with the parent component. QS21 has been shown to activate MHC class I restricted CTLs to several subunit Ags, as well as to stimulate Ag specific lymphocytic proliferation (Kensil, 1992). Aquila (formally Cambridge Biotech Corporation) produces and supplies QS21 to SB-Biologicals.

Experiments performed at SmithKline Beecham Biologicals have demonstrated a clear synergistic effect of combinations of MPL and QS21 in the induction of both humoral and TH1 type cellular immune responses.

The oil/water emulsion is composed an organic phase made of of 2 oils (a tocopherol and squalene), and an aqueous phase of PBS containing Tween 80 as emulsifier. The emulsion comprised 5% squalene 5% tocopherol 0.4% Tween 80 and had an average particle size of 180 nm and is known as SB62 (see WO 95/17210).

Experiments performed at SmithKline Beecham Biologicals have proven that the adjunction of this O/W emulsion to 3D-MPL/QS21 (SBAS2) further increases the immunostimulant properties of the latter against various subunit antigens.

2.—Preparation of Emulsion SB62 (2 Fold Concentrate):

Tween 80 is dissolved in phosphate buffered saline (PBS) to give a 2% solution in the PBS. To provide 100 ml two fold concentrate emulsion 5 g of DL alpha tocopherol and 5 ml of squalene are vortexed to mix thoroughly. 90 ml of PBS/Tween solution is added and mixed thoroughly. The resulting emulsion is then passed through a syringe and finally microfluidised by using an M110S microfluidics machine. The resulting oil droplets have a size of approximately 180 nm.

3.—Preparation of Xenogeneic or Human P501S QS21/3D MPL Oil in Water (SBAS2) Formulation:

The adjuvant is formulated as a combination of MPL and QS21, in an oil/water emulsion. The formulations are prepared extemporaneously on the day of injection.

The formulations containing 3D-MPL and QS21 in an oil/water emulsion (SBAS2B formulations) are performed as follows: xeno or human P501S (20 μg) is diluted in 10-fold concentrated PBS pH 6.8 and H₂O before consecutive addition of SB62 (50 μl), MPL (20 μg), QS21 (20 μg) and 1 μg/ml thiomersal as preservative at 5 min intervals. All incubations are carried out at room temperature with agitation.

The non-adjuvanted formulations are performed as follows: recombinant xeno P501S (20 μg) is diluted in 1.5 M NaCl and H₂O before addition of 1 μg/ml thiomersal as preservative at 5 min intervals. All incubations are carried out at room temperature with agitation.

B—Immunogenicity Experiments

B-1 Immunisation Protocol with a Protein-Based Approach

A xenogeneic antigen to human P501S can be used, according to the present invention, to induce an immune response against a closely related autologous tumour antigen. Similarly a human P501S can be used to immunise animal species and assess the level of cross-reacting antibodies. The quality and the intensity of the immune response induced by different molecules can be compared as well as the capacity of this immune response to cross-react with other forms of the P501S protein. The protein can be adjuvanted or not.

Rabbits were vaccinated three times, intramuscularly, at 3 weeks interval with 100 μg of human P501S formulated or not in SBAS02 (see above). Three weeks after the third injection, blood can be taken and the sera tested for the presence of anti-P501S antibodies.

The anti-P501S antibody response (Total IgG Antibody response) is classically assessed by ELISA, using purified human P501S protein as a coating antigen.

Spleen and lymph nodes of these immunized animals can also be used to analyze the cellular immune responses induced by the vaccinations. Lymphoproliferative responses can be evaluated after 72 hours of in-vitro re-stimulation with the different forms of the molecules used to vaccinate, or with the purified human P501S protein.

B-2 Immunisation Protocol with a DNA-Based Approach

1. Protocol

Female or male C57BL/6 mice were immunised with a P501S DNA construct by gene gun or PMID (particle mediated intradermal delivery). The DNA construct is labelled JNW680 and comprises the coding sequence of the full-length human P501S gene (Genbank data base accession number AY033593) cloned into a standard eukaryotic expression vector pVAC1 (Thomsen Immunology 95:510P106, 1998). Plasmid DNA was precipitated onto 2 μM golds beads using calcium chloride and spermidine. Loaded beads were coated onto Tefzel tubing as described (Eisenbaum et al. 1993, Pertmer et al. 1996). Particle bombardment was performed using the Accell gene dlivery system (WO95/19799). Each administration consisted of two bombardments with DNA/gold providing a total dose of approximately 1-5 μg of plasmid DNA. Mice were routinely immunised on day 0, 21, 42 & 63.

2. Read-Outs

Cellular responses were monitored by IFNγ/IL-2 ELISPOT using splenocytes harvested 7 days post each immunisation. Splenocytes were re-stimulated using peptides identified from a peptide library covering a majority of the P501S sequence.

Antibody responses were monitored from serum samples taken at the mice were sacrificed. Antibody responses were assessed by ELISA using CPC-P501S to coat the plates.

3. Results

3.1 Peptide Library

Following immunisation of female mice with the human P501S construct, individual P501S peptides were used to re-stimulate the splenocytes in an IFNγ/IL-2 ELISPOT. From this library screen, three peptides were identified which induced either IL-2 and/or IFNγ. These peptides were labelled peptides 18, 22 and 48. Further studies have identified that peptides 22 and 48 contain CD4 epitopes.

The sequences of the peptides as follows:

| | |
|---|---|
| Peptide 18: | HCRQAYSVYAFMISLGGCLG |
| Peptide 22: | GLSAPSLSPHCCPCRARLAF |
| Peptide 48: | VCLAAGITYVPPLLLEVGV |

3.2 Confirmation of Responses to these Peptides

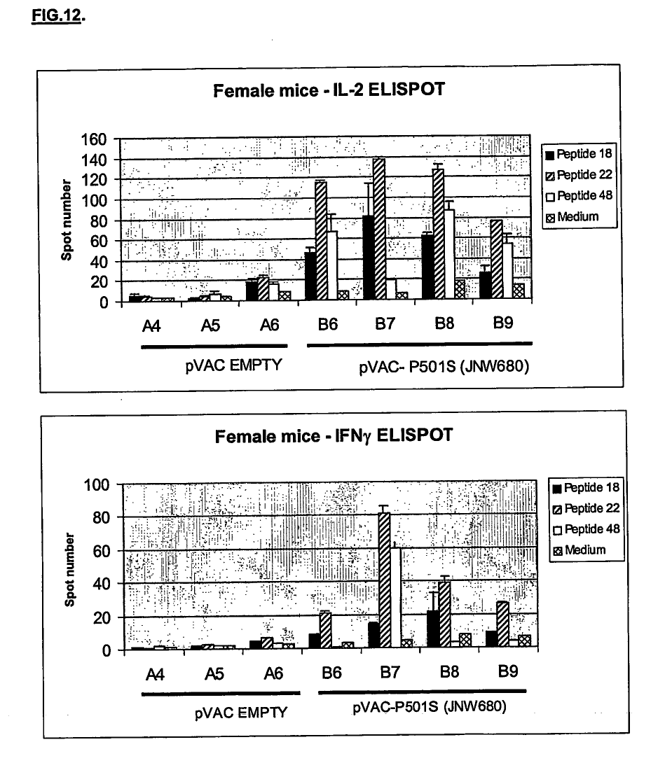

In independent experiments, the induction of an immune response to these peptide epitopes was confirmed in both male and female mice following PMID immunisation with the P501S construct (JNW680). FIGS. 11 and 12 show that good IL2 and/or IFNγ responses were induced in a majority of mice for male and female mice respectively to all three peptides, whereas mice immunised with an empty vector generated no specific responses.

3.3 Alignment of Peptides

The table 1 below shows the number and positions of amino acids which differ between the human and mouse P501S sequence in the regions encoded by Peptides 18, 22 and 48.

TABLE 1

| | | |
|---|---|---|
| | Sequence | |
| | (differences between human and | No. of |
| Peptide | mouse are boxed) | changes |

| | | |
|---|---|---|
| 18 | HCRQA\|\overline{Y}\|SVYAFMISLGGCLG | 1 |
| 22 | GL\|\overline{SAPSL}\|S\|\overline{PH}\|CCPC\|\overline{RAR}\|LAF | 10 |
| 48 | VCLAAGITYVPPLLLEVGV | 0 |

4. Conclusions

Responses to the epitope encoded by Peptide 48 were detected in both female and male mice. Comparison of the human and mouse sequence in this region confirms that there is 100% sequence identity. Therefore one conclusion of this study is that human P501S can be used to induce immune responses which have the potential to be cross-reactive with the mouse P501S. Therefore one can reasonably assume, that for the reason given above (sequence identity), mouse P501S has the potential to induce immune responses which are cross-reactive with the human P501S, validating the xenogeneic approach for this antigen.

### EXAMPLE IV

Analysis of P501S Expression by Real-Time PCR

1.—Introduction

Expression analysis of the P501S will be done in animal models and in animal cell lines by monitoring the P501S mRNA abundance by real-time PCR.

Animal models are used to test vaccine composition and to evaluate their immunogenicity (ex: specific CTL induction) and their potential toxicity (ex: autoimmunity). The more relevant animal model will display a tissue expression pattern of the P501S, which is the closest to the human profile. Expression level measurement will be done in animal prostate and in a panel of essential tissues. Real-time PCR is also used to characterise the expression level of the P501S gene in animal cell lines such as rat prostate cell lines (CRL-2275, CRL-2276). Objective being to identify animal cell lines, which are expressing P501S at a level, which is closest to the level observed in human prostate tumours. Animal cell lines identified to express reasonable level of P501S mRNA could be used to establish an animal tumour model. Anti-tumour effects of vaccination using the P501S-purified protein in adjuvant could be monitored either by tumour regression or by protection against tumour challenge in the animal.

2.—Real-Time RT-PCR Analysis

Real-time RT-PCR (U. Gibson. 1996. Genome Research: 6, 996) is used to compare mRNA transcript abundance of the target protein in a panel of tissues and cell lines.

Total RNA is extracted from snap frozen biopsies using TriPure reagent (Boehringer). Poly-A+ mRNA is purified from total RNA after DNAase treatment using oligo-dT magnetic beads (Dynal). Quantification of the mRNA is performed by spectrofluorimetry (VersaFluor, BioRad) using RiboGreen (Molecular Probes). Primers for real-time PCR amplification are designed with the Perkin-Elmer Primer Express software using default options for TaqMan amplification conditions.

Real-time reactions are assembled according to standard PCR protocols using 2 ng of purified mRNA for each reaction. Real-time PCR amplification are monitored using a Taqman probe. Amplification (40 cycles) and real-time detection is performed in a Perkin-Elmer Biosystems PE7700 system using conventional instrument settings. Ct values are calculated using the PE7700 Sequence Detector Software. Ct values are obtained from each tissue sample for the target mRNA (CtX) and for the beta actin mRNA (CtA).

As the efficiency of PCR amplification under the prevailing experimental conditions is close to the theoretical amplification efficiency, 2^{(CtA-CtX)} value is an estimate of the relative target transcript level in the sample, standardized with respect to Actin transcript level. A value of 1 thus suggests that the candidate antigen and Actin have the same expression level.

For the rat model, real-time (RT) PCR reactions were performed on 2 rat prostate cell lines (CRL-2222 and CRL-2276) and on a panel of 11 rat tissues such as brain, colon, femur, gum, heart, kidney, liver, lung, prostate, spleen, testis.

For the Cynomolgus model, expression of P501 homologue was evaluated in prostate.

For the mouse model, expression level was determined in prostate, colon, lung, brain, kidney, spleen, tests, stomach, heart and liver.

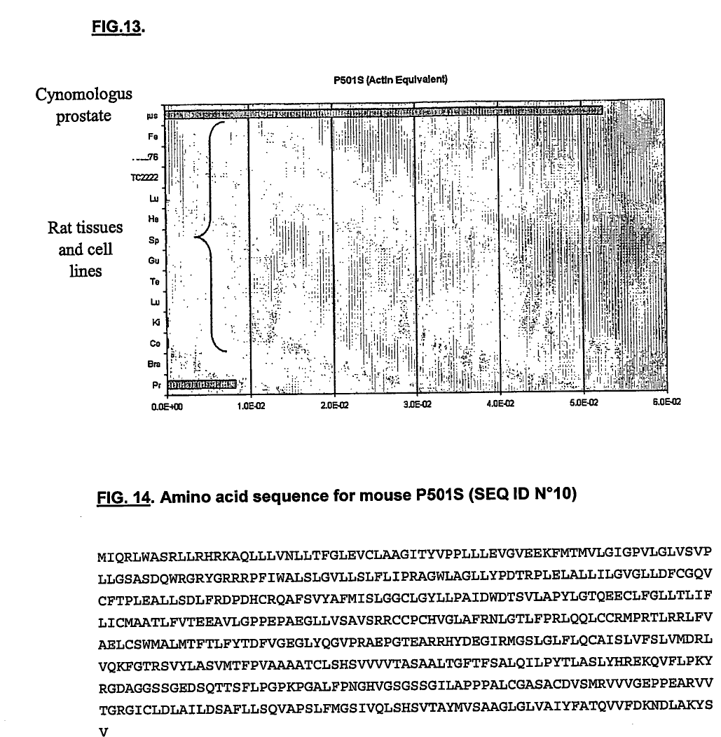

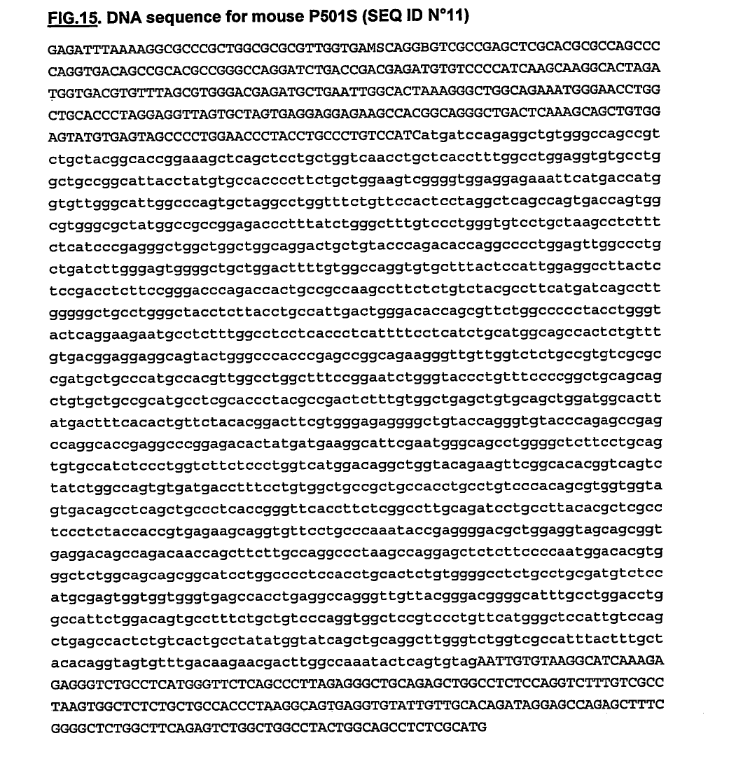

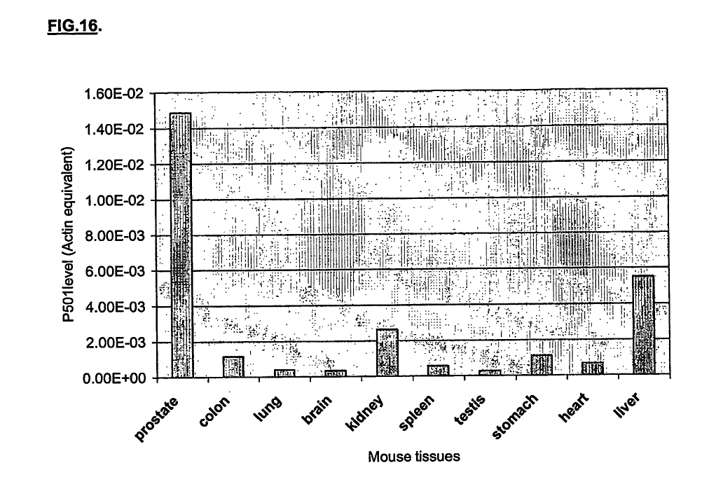

P501 homologue transcript level are calculated as described above. Results are shown in Table 2, Table 3, Table 4 and FIGS. 13 and 16.

TABLE 2
RT-PCR analysis of P501S on a panel of rat tissues and rat cell lines.
P501 expression analysis in rat tissues

| | | CT of | CT of | P501S |
|---|---|---|---|---|
| Tissue | Abbreviation | P501S | actin | Actin |
| prostate | Pr | 26 | 19 | 8.2E−03 |
| brain | Bra | 34 | 19 | 3.1E−05 |
| colon | Co | 31 | 18 | 1.4E−04 |
| kidney | Ki | 31 | 19 | 1.4E−04 |
| lung | Lu | 35 | 18 | 1.4E−05 |
| testis | Te | 37 | 20 | 9.3E−06 |
| gum | Gu | 32 | 18 | 7.9E−05 |
| spleen | Sp | 30 | 17 | 9.7E−05 |
| heart | He | 35 | 21 | 5.5E−05 |
| liver | Lu | 33 | 20 | 9.2E−05 |
| tumor cell 2222 | TC2222 | 34 | 17 | 8.3E−06 |
| tumor cell 2276 | TC2276 | 40 | 17 | 8.7E−08 |
| femur | Fe | 31 | 17 | 6.3E−05 |

TABLE 3
RT-PCR analysis of P501S on Cynomolgus prostate.
P501 expression analysis in Cynomolgus prostate

| | | CT of | CT of | P501S |
|---|---|---|---|---|
| Tissue | Abbreviation | P501S | actin | Actin |
| prostate | Pr | 24 | 19 | 5.3E−02 |

TABLE 4
RT PCR analysis of P501S on a panel of 10 mouse tissues
P501 expression analysis in mouse tissues

| | Exp. 1 | | Exp. 2 | | Exp. 3 | | |
|---|---|---|---|---|---|---|---|
| | CT of | CT of | CT of | CT of | CT of | CT of | P501S |
| Tissue | P501S | actin | P501S | actin | P501S | actin | Actin |
| prostate | 24 | 19 | 27 | 20 | 26 | 20 | 1.5E−02 |
| colon | 27 | 18 | 30 | 19 | 31 | 21 | 1.2E−03 |
| lung | 28 | 18 | 33 | 20 | 33 | 21 | 3.9E−04 |
| brain | 30 | 20 | 34 | 21 | 36 | 23 | 3.3E−04 |
| kidney | 28 | 19 | 31 | 22 | 31 | 22 | 2.6E−03 |
| spleen | 29 | 18 | 31 | 20 | 31 | 20 | 5.6E−04 |
| testis | 29 | 18 | 32 | 19 | 33 | 20 | 2.7E−04 |
| stomach | 28 | 19 | 31 | 21 | 31 | 22 | 1.1E−03 |
| heart | 32 | 21 | 33 | 23 | 35 | 24 | 6.7E−04 |
| liver | 29 | 21 | 31 | 23 | 32 | 24 | 5.5E−03 |

P501S is expressed in rat, Cynomolgus and mouse prostate (0.8%, 5.3% and 1.5% relative to actin level, respectively). Average P501 trancript level in rat other tissues (0.007%) is hundred fold lower than in rat prostate. No significant expression was detected in both rat cell lines. In other mouse tissues, the highest expression level was detected in the liver and in the kidney (3 and 6 times lower than in mouse prostate, respectively).

### EXAMPLE V

Induction of P501S-Specific CD4 or CD8 T Cells by Xenogeneic P501S

A T-cell in vitro priming protocol is used to demonstrate the capacity of the human immune repertoire to recognize the P501S protein as a potential target for immunotherapy. This protocol can be used to generate and expand either CD4 or CD8 human T cells that specifically recognise either the P501S-derived peptide or the P501S protein loaded onto targets but also human cells that endogeneously express the P501S.

The protocol used to generate P501S specific CD8 T cells is briefly described: Human dendritic cells (DC) genetically engineered to express the xenogeneic P501S gene or pulsed with 1 μg/ml xenogeneic P501S-derived peptides, are matured for 48 hours using CD40L, and cultured with autologous PBMC in medium supplemented with IL-7. Weekly stimulations are performed using adherent PBMC pulsed with 1 μg/ml xenogeneic P501S, with the addition of IL-7 on day 0 and 4, and IL-2 on days 1 and 4. Lines are assayed following the 4^{th}, 5^{th}, and 6^{th} round of stimulation by ELISPOT assays to measure IFNg secretion. Antigen presenting cells (APC) in the ELISPOT assays are autologous B-LCL, pulsed either with the xenogeneic P501S or an irrelevant peptide. Specific CTL activity is initially detectable after the 5^{th} or the 6^{th} stimulation cycles against xenogeneic P501S pulsed or transduced APC. A similar protocol can be used to generate P501S specific CD4 T cell clones.

### EXAMPLE VI

The induction of a more potent immune response by using a xenogeneic protein is shown by vaccinating mice, rat, monkey, and human with a human protein and by reading the antibody response by ELISA. In this experiment, mice and non-humpan primate are vaccinated with the recombinant human P501S antigen delivered as CPC-P501 protein+adjuvant, CPC-P501S-encoding adenoviral vector, or CPC-P501S-encoding DNA. The sera of these animals are collected after each vaccination and the antibody titers are assessed by standard ELISA using coated human P501S or CPC. The dilution of each sera is adjusted to show a similar signal by ELISA when read against CPC, then, at the very similar dilution the signal is read with an ELISA using the coated-human P501. This experiment shows that in primate the human P501 vaccine induces higher titers of antibodies after a limited number of injections, while in mice the anitbody response is more potent. This experiment demonstrates that the xenogenic vaccination in mouse model (the mouse P501S is 90.8% identical to the human P501S at the amino acid sequence) the induction of the immune response is more efficient than in the non-human primate model close to the syngenic situation (the cynomolgous monkey P501S is 98.0% identical to the human P501S at the amino acid sequence).

### EXAMPLE VII

Characterisation of P501S Specific Antibodies Induced by Xenogeneic P501S

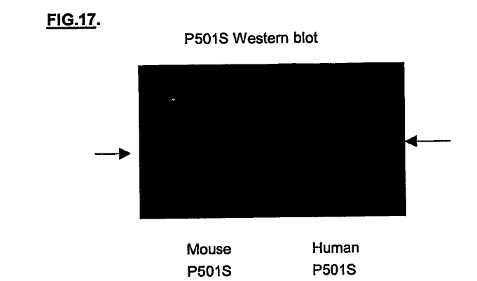

The induction of a cross-reactive antibody response following immunisation with either mouse, monkey or rat with human P501S adjuvanted protein or DNA is investigated using an in vitro Western blot assay or ELISA. In this experiment a mammalian expression vector is constructed in which the mouse, monkey or rat DNA sequence is inserted downstream of a CMV promoter. Upon transfection of these DNA vectors into a host cell line (such as CHO or COS cells), the mouse, monkey or rat P501S gene is expressed. A whole cell lysate from these cells is used in a Western blot. For the mouse expression vector, mouse P501S coding sequence (SEQUENCE ID NO:11) was engineered using overlapping PCR methodology and cloned into the pVAC1 expression vector. In the case of the mouse P501S expression vector, expression was confirmed in a Western blot using a rabbit anti-P501S polyclonal sera (FIG. 17). On the other hand, for the ELISA, the plates are coated with a self polypeptide-coated in the plates, such as a peptide from amino acid 296 to 322 that shows 100% identity between human and mouse and is a B-cell epitope recognized by a monoclonal antibody generated against P501S.

A Western blot using the whole cell lysate from cells transfected with either mouse, monkey or rat P501S is used to confirm the presence of cross-reactive P501S specific antibodies. In this case, sera is taken from mice, monkeys or rats previously immunised with human P501S or human P501S fusion proteins. This sera may be used at a dilution of 1:10-1:100,000 in a Western blot protocol, using a relevant secondary antibody conjugated to horse raddish peroxidase (HRP). A stronger signal at equivalent dilution or higher dilution at equivalent signal on the Western blot after blotting with the immune sera obtained from a xenogeneic vaccination than a syngeneic vaccination demonstrates that the xenogeneic vaccination is able to induce a stronger immune response recognising the antigen. A similar result by using the ELISA leads to same conclusion.

1. An immunogenic composition comprising a xenogeneic P501S polypeptide or a xenogeneic P501S-encoding polynucleotide, or an immunogenic fragment thereof; and a pharmaceutically acceptable carrier.

2. An immunogenic composition as claimed in claim 1 wherein the xenogeneic P501S polypeptide or immunogenic fragment thereof comprises SEQ ID NO:1 or SEQ ID NO:3 or SEQ ID NO:10.

3. An immunogenic composition as claimed in claim 1 wherein the xenogeneic P501S-encoding polynucleotide or immunogenic fragment comprises SEQ ID NO:2 or SEQ ID NO:4 or SEQ ID NO:1.

4. An immunogenic composition as claimed in claim 1 which additionally comprises a TH-1 inducing adjuvant.

5. An immunogenic composition as claimed in claim 4 in which the TH-1 inducing adjuvant comprises 3D-MPL, QS21, an immunostimulatory CpG oligonucleotide, a mixture of QS21 or cholesterol.

6. An immunogenic composition comprising an effective amount of antigen presenting cells, modified by in vitro loading with a xenogeneic P501S polypeptide or immunogenic fragment thereof, or genetically modified in vitro to express a xenogeneic P501S polypeptide and a pharmaceutically effective carrier.

7. A pharmaceutical composition comprising the immunogenic composition as claimed in claim 1.

8. A process for the production of an immunogenic composition as claimed in claim 1, comprising admixing a xenogeneic P501S polypeptide or a xenogeneic P501S-encoding polynucleotide with a suitable adjuvant, diluent or other pharmaceutically acceptable carrier.

9. An isolated polypeptide comprising an amino acid sequence which has at least 92% identity to the amino acid sequence of SEQ ID NO:1 over the entire length of SEQ ID NO:1.

10. An isolated polypeptide as claimed in claim 9 in which the amino acid sequence has at least 95% identity to SEQ ID NO:1.

11. The polypeptide as claimed in claim 10 comprising the amino acid sequence of SEQ ID NO:1.

12. The isolated polypeptide of SEQ ID NO:1.

13. A polypeptide comprising an immunogenic fragment of a polypeptide as claimed in claim 9 in which the immunogenic activity of the immunogenic fragment is substantially the same as the polypeptide of SEQ ID NO:1.

14. A polypeptide as claimed in claim 9 wherein said polypeptide is part of a larger fusion protein.

15. An isolated polynucleotide encoding a polypeptide as claimed in claim 9.

16. The isolated polynucleotide of claim 15, comprising the sequence of SEQ ID NO:2.

17. An isolated polynucleotide comprising a nucleotide sequence encoding a polypeptide that has at least 92% identity to the amino acid sequence of SEQ ID NO:2, over the entire length of SEQ ID NO:2; or a nucleotide sequence complementary to said isolated polynucleotide.

18. The isolated polynucleotide of claim 15 in which the identity of said polynucleotide to SEQ ID NO:1 is at least 95%.

19. An expression vector or a recombinant live microorganism comprising an isolated polynucleotide according to claim 15.

20. A host cell comprising the expression vector of claim 19.

21. A process for producing a polypeptide of claim 9 comprising culturing a host cell comprising a polynucleotide comprising a nucleotide sequence encoding a polypeptide that has at least 92% identity to the amino acid sequence of SEQ ID NO:2, over the entire length of SEQ ID NO:2; or a nucleotide sequence complementary to said isolated polynucleotide under conditions sufficient for the production of said polypeptide and recovering the polypeptide from the culture medium.

22. An immunogenic composition for immunotherapeutically treating a patient suffering from or susceptible to prostate cancer or other P501S-associated tumours or diseases comprising a polypeptide of claim 9.

23. A method of inducing an immune response against human P501S having an amino acid sequence as set forth in SEQ ID NO:5 to SEQ ID NO:7 in a human, comprising administering to the subject an effective dosage of an immunogenic composition comprising a xenogeneic form of said human P501S.

24. The method of claim 23, wherein said immunogenic composition comprises a xenogenic P501S polypeptide or a fragment thereof.

25. The method of claim 23, wherein said xenogeneic form of human P501S is the rat P501S, which has at least 92% identity to the amino acid sequence of SEQ ID NO:1 over the entire length of SEQ ID NO:1

26. The method of claim 23, wherein said xenogeneic form of human P501S is selected from the group consisting of the mouse P501S having the sequence as set forth in SEQ ID NO:10 and the Cynomolgus monkey P501S having the sequence set forth in SEQ ID NO:3.

27. The method of claim 23, wherein said immunogenic composition includes a live viral expression system or a plasmid vector which expresses said xenogeneic antigen, through antigen loaded dendritic cells.

28. The method of claim 23, wherein said immunogenic composition comprises a xenogenic P501S-encoding polynucleotide or a fragment thereof.


SEQUENCE LISTING (11 sequences)

SEQ ID NO: 1 - protein, 553 aa, Rattus norvegicus
MIQRLWASRLLRHRKAQLLLVNLLTFGLEVCLAAGITYVPPLLLEVGVEEKFMTMVLGIGPVLGLVSVPLLGSASDQWRGRYGRRRPFIWALSLGVLLSLFLIPRAGWLAGLLCSDTRPLELALLILGVGLLDFCGQVCFTPLEALLSDLFRDPDHCRQAFSVYAFMISLGGCLGYLLPAIDWDTSALAPYLGTQEECLFGLLTLIFLICVAATLLVAEEAVLGPPEPAEGLLVSSVSRRCCSCHAGLAFRNLGTLFPRLHQLCCRMPRTLRRLFVAELCSWMALMTFTLFYTDFVGEGLYQGVPRAEPGTEARRHYDEGIRMGSLGLFLQCAISLFFSLVMDRLVQKFGTRSVYLASVMTFPVAAAATCLSHSVVVVTASAALTGFTFSALQILPYTLASLYHREKQVFLPKYRGDAGGGSSEDSQTTSFLLGPKPGAPFPNGHVGPGGSSILVPPPALCGASACDVSMRVVVGEPPEAKVVTGRGICLDLAILDSAFLLSQVAPSLFMGSIVQLSHSVTAYMVSAAGLGLVAIYFATQVVFDKNDLAKYSL

SEQ ID NO: 2 - DNA, 2314 bp, Rattus norvegicus
gggctcttagacaccgcaacaaaagcaactttcctccaagccactgccacctgttgggttttcacacatttcgagctttagttccgatccccagaacatccacgtagtttttctggccttctggctgagccatggaggccgacagaggaggggagaagtttgaagcttgagaaggatttccgtatgcgcaaggctacccatgcttgtccttcctcccatgaccctggtcagccctcctctgccctcctcttcctgccccccttctctccagggtccgactgacgagatgtgtccccatcaagcaaggcactagatggtgacgtgttcagtgtgggatgagatgccgaagtggtactcaagggctggccgaaatgggagcctggctgcaccctcggaggttggtgctagcaaggaggagaagccgcggcagggctgactcaaaacagctgtggggtgtgtgaatggcccccggacccctaaccgccctgtccatcatgatccagaggctgtgggccagccgtctgctaaggcatcggaaagcccagctcctgctggtcaacctgctaaccttcggcctggaggtgtgcctggctgctggcattacctatgtgccaccccttctgctggaagtcggggtagaggaaaagttcatgaccatggtgttgggcattggcccagtgctgggcctggtttctgttccactcctaggctcagccagtgaccagtggcgtgggcgctatggccgccggagaccctttatctgggctctgtccctgggtgtcctgctaagcctcttcctcatcccgagggccggctggctggcagggctactgtgttcagatactaggcccctggagttggccctgctcatcttgggagtggggctgctggacttttgcggccaggtgtgctttactccactggaggccttactctccgacctcttccgggacccagaccactgccgccaagccttctctgtctatgccttcatgatcagcctcgggggctgcctgggctacctcttacctgccattgactgggacaccagcgccctggccccctacctaggcactcaggaagaatgcctcttcggcctcctcaccctcatttttctcatctgtgtggcagccactctgcttgtggctgaggaggcagtccttggcccacccgagccagcagaagggttgttggtctcctccgtgtcacgccggtgctgctcatgccatgctggcctggctttccggaatctgggtaccctgtttccccggctgcaccagctgtgctgccgaatgcctcgcaccctgcgccggctctttgtggctgagctgtgcagctggatggcacttatgactttcacactgttctacacggacttcgtgggagaggggctgtaccagggtgtccccagagcagagccaggtaccgaggcccggagacactatgatgaaggcattcgaatgggcagcctggggctcttcctgcagtgtgccatctccctgttcttctccctggtcatggacaggctggtacagaagttcggcacacggtcagtctacctggccagtgtgatgacctttcccgtggctgccgctgccacgtgcctgtcccacagcgtggttgtagtgacagcctcagctgccctcaccgggttcaccttctcagccttgcagatcctgccttacacgctcgcctccctctaccatcgagagaagcaggtgttcctgcccaaataccgaggggacgctggaggtggtagcagtgaagacagccaaacaaccagcttcttgctaggccctaagccaggagctcccttccccaatggacacgtgggccctggcggcagcagcatcctggtgcccccacctgcactctgtggggcctctgcctgtgatgtctccatgcgagtggtagtgggtgagccacctgaagccaaggttgttactggacggggcatttgcctggaccttgccatcctggacagtgcctttctgctgtcccaggtggctccgtccctgttcatgggctccattgtccagctgagccactctgtcactgcctatatggtatcagctgcaggcttgggtctggtcgccatttactttgctacacaggtagtgtttgacaagaatgacttggccaaatactcactgtagaattctgtaaggcatcaaagaagaggatctgcctccccggttctcagccccagagggctgcagagctggtctctttccggtctctgttgccctgagtggctctccactgccatccgaaggcagtgaggtgtatggctgcacaggttggagcttttggc

SEQ ID NO: 3 - protein, 553 aa, Maccaca fascicularis
MVQRLWVSRLLRHRKAQLLLINLLTFGLEVCLAAGITYVPPLLLEVGVEEKFMTMVLGIGPVLGLVSVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPAEPAEGLSAPSLPSHCCPCWARLAFRNLGALLPRLHQLCCRMPRTLRRLFVAELCSWMALMTFTLFYTDFVGEGLYQGVPRAELGTEARRHYDEGVRMGSLGLFLQCAISLVFSLVMDRLVQRFGTRAVYLASVAAFPVAAGATCLSHSVAVVTASAALTGFTFSALQILPYTLASLYHRERQVFLPKYRGDAGGTSSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSV

SEQ ID NO: 4 - DNA, 3514 bp, Maccaca fascicularis
aaaaaaaaagccgccggctggcgcgcgtggggggcaaggaaaagaggggggaaaccagtctgcacgcgctggctccgggtgacagccgcgcgcctaggccaggcagcgtctccctctgtcacccagactggaggcaatgttctgatcactgcacactgcacccttgacctcccagactcaagcaatcctcccatctcagcctcttaagtagctgggaccacaggatctgagtgatgagatgtgtccccactgaggtgccccacagcagcaggtgttgagcatgggctgagaagctggaccggcaccaaagggctggcggaaatgggcgcctggctgattcctaggcagttggcggcagcaaggaggagaggccgtggcttccggagcagagcggagacgaagcagttctggagtgcttaaacggccccctgagccctacccgcctggcccactatggtccagaggctgtgggtgagccgcctgctgcggcaccggaaagcccagctcttgttgatcaacctgctaacctttggcctggaagtgtgtttggccgcaggcatcacctatgtgccgcctctgctgctggaagtgggggtagaagagaagttcatgaccatggtgctgggcatcggtccagtgctgggcctggtctctgtcccactcctaggctcagccagtgaccactggcgcggacgctatggccgccggcggcccttcatctgggcgctgtccttgggcatcctgctgagcctctttctcatcccaagggctggctggctggcagggctgctgtgcccggatcccaggcccctggagctggcactgctcatcctgggcgtagggctgctggacttctgtggccaggtgtgcttcactccactggaggccctgctctctgacctgttccgggacccggaccactgtcgccaggcctactccgtctatgccttcatgatcagtcttgggggctgcctgggctacctcctgcctgccattgactgggacaccagtgccctggccccctacctgggcacccaggaggagtgcctctttggcctgctcaccctcatcttcctcacctgcgtagcagccacactgctggtggccgaggaggcagcactgggccccgccgagccagcggaagggctgtctgccccctccctgccgtcccactgctgtccgtgctgggcccgcctggctttccggaacctgggcgccctgcttccccggctgcaccagctgtgctgccgcatgccccgcaccctgcgccggctcttcgtggctgagctgtgcagctggatggcactcatgaccttcacgctgttttacacggatttcgtgggcgaggggctataccagggcgtgcccagagctgagctgggcaccgaggcccggagacactatgatgaaggcgttcggatgggcagtctggggctgttcctgcagtgcgccatctccctggtcttctctctggtcatggaccggctggtgcagcgattcggcactcgagcagtctatctggccagtgtggcagctttccctgtggctgccggtgccacgtgcctgtcccacagtgtggctgtggtgacggcttcagccgccctcactgggttcaccttctcagccctgcagatcctgccctacacactggcctccctctaccaccgggagaggcaggtgttcctgcccaaataccgaggggacgctggaggcactagcagtgaggacagcctgatgactagcttcctgccaggccctaagcctggagctcccttccctaatggacacgtgggtgctggaggcagtggcctgcttccacctccacccgcgctctgcggggcctctgcctgcgatgtctctgtacgtgtggtggtgggtgagcccaccgaggccagggtggttccgggccggggcatctgcctggacctcgccatcctggatagtgccttcctgctgtcccaggtggccccgtccctgttcatgggctccatcgtccagctcagccagtctgtcactgcctatatggtgtctgctgcaggcctgggtctggttgccatttactttgctacacaggtagtatttgacaagagcgacttggccaaatactcggtgtagaaaacttccagcacattggggtggagggcctgcctcactgggtcccagctccccactctttgttagccccatggggctgctgggctggccgccagtttctgttgctgccaaagtaatgtggctctctgctgccaccctgtgctgctgaggtgcgtagctgcacagctgggggctggggcatccctctccctcctccccagtctctagggctgcctgactggaagccttccaagggggtttcagtctggacttctacagggaggctagaagggcagggcatttgattcgctccatgcactggaatgtggggactctgcaggtggattacccaggctcagggttaacagctagcctcctggctgagacatacctagagaaggggttttgggagctgagtaaactcagtcacctggtttcccacctctaagcccccttaacctgcagcttcatttaatgtagctcttgcatgggagtttctaggatgaaacactcctccgtgggatttgaacgtatgaaagttatttgtaggggaagagtcctgaggggcaacacaccaggtcccctcagcccacagcactgcctttttgctgatcccctgactcttaccttttatcaggacgtggcctattggtccctttgttgccatcatagggacacaggcatttaaatatttaacttatttatttaacaaagtagaagggaatccattgctagcttttgtgtgttggtgtctaagatttgggtagggtgggatccccaacaatcaggtccactgagatcactggtcattgggctgatcattgccagaatcttcttctcctggggtctggctcctcaaaatgcctaacccaggaccttggaaattttactcatcccgactgataattccaaatgctgttacccaaggttagggggttgaaggaaggtggagggtggggcttcaggtctcaacagcttccctaaccaccccttttctcttggcccagcctggttccccccacttctactcccctctactgtctctaggactgggctgatgaaggcactgcctgaaatttccctcacccccaactttccccactggctccacaaccctgtttggagctgttgcaggaccagaagcacaaagtgtggtttcccaggcctttgtccatctcagccccccagagtatatctgtgcttggggaatctcacacagaaactcaggagcaccccctgcctgagctaaggaggtcttatctctcaggggggtttaagtgccgtttgcaataatgtcttatttatttagcggggcaaatattttatactgtaagtgagcaatcagtataatgtttatggtgatgaaattaaaggctttcttatatgtttaaaaaaaaaaaaaaaaaaaaaaaaaaaaaaa

SEQ ID NO: 5 - protein, 553 aa, Homo sapiens
MVQRLWVSRLLRHRKAQLLLVNLLTFGLEVCLAAGITYVPPLLLEVGVEEKFMTMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSA

SEQ ID NO: 6 - protein, 255 aa, Homo sapiens
GLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSA

SEQ ID NO: 7 - protein, 231 aa, Homo sapiens
MGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAALGLVAIYFATQVVFDKSDLAKYSA

SEQ ID NO: 8 - DNA, 1788 bp, Artificial Sequence
  DNA sequence for alphaprepro signal sequence fused to human P501S and fused to a His tag
atgagtttcctcaattttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagccatggtgctgggcattggtccagtgctgggcctggtctgtgtcccgctcctaggctcagccagtgaccactggcgtggacgctatggccgccgccggcccttcatctgggcactgtccttgggcatcctgctgagcctctttctcatcccaagggccggctggctagcagggctgctgtgcccggatcccaggcccctggagctggcactgctcatcctgggcgtggggctgctggacttctgtggccaggtgtgcttcactccactggaggccctgctctctgacctcttccgggacccggaccactgtcgccaggcctactctgtctatgccttcatgatcagtcttgggggctgcctgggctacctcctgcctgccattgactgggacaccagtgccctggccccctacctgggcacccaggaggagtgcctctttggcctgctcaccctcatcttcctcacctgcgtagcagccacactgctggtggctgaggaggcagcgctgggccccaccgagccagcagaagggctgtcggccccctccttgtcgccccactgctgtccatgccgggcccgcttggctttccggaacctgggcgccctgcttccccggctgcaccagctgtgctgccgcatgccccgcaccctgcgccggctcttcgtggctgagctgtgcagctggatggcactcatgaccttcacgctgttttacacggatttcgtgggcgaggggctgtaccagggcgtgcccagagctgagccgggcaccgaggcccggagacactatgatgaaggcgttcggatgggcagcctggggctgttcctgcagtgcgccatctccctggtcttctctctggtcatggaccggctggtgcagcgattcggcactcgagcagtctatttggccagtgtggcagctttccctgtggctgccggtgccacatgcctgtcccacagtgtggccgtggtgacagcttcagccgccctcaccgggttcaccttctcagccctgcagatcctgccctacacactggcctccctctaccaccgggagaagcaggtgttcctgcccaaataccgaggggacactggaggtgctagcagtgaggacagcctgatgaccagcttcctgccaggccctaagcctggagctcccttccctaatggacacgtgggtgctggaggcagtggcctgctcccacctccacccgcgctctgcggggcctctgcctgtgatgtctccgtacgtgtggtggtgggtgagcccaccgaggccagggtggttccgggccggggcatctgcctggacctcgccatcctggatagtgccttcctgctgtcccaggtggccccatccctgtttatgggctccattgtccagctcagccagtctgtcactgcctatatggtgtctgccgcaggcctgggtctggtcgccatttactttgctacacaggtagtatttgacaagagcgacttggccaaatactcagcgggtggacaccatcaccatcaccattaa

SEQ ID NO: 9 - protein, 595 aa, Artificial Sequence
  Polypeptide sequence for alphaprepro signal sequence fused to human P501S and fused to a His tag
MSFLNFTAVLFAASSALAAPVNTTTEDETAQIPAEAVIGYSDLEGDFDVAVLPFSNSTNNGLLFINTTIASIAAKEEGVSLEKREAEAMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSAGGHHHHHH

SEQ ID NO: 10 - protein, 553 aa, Mus musculus
MIQRLWASRLLRHRKAQLLLVNLLTFGLEVCLAAGITYVPPLLLEVGVEEKFMTMVLGIGPVLGLVSVPLLGSASDQWRGRYGRRRPFIWALSLGVLLSLFLIPRAGWLAGLLYPDTRPLELALLILGVGLLDFCGQVCFTPLEALLSDLFRDPDHCRQAFSVYAFMISLGGCLGYLLPAIDWDTSVLAPYLGTQEECLFGLLTLIFLICMAATLFVTEEAVLGPPEPAEGLLVSAVSRRCCPCHVGLAFRNLGTLFPRLQQLCCRMPRTLRRLFVAELCSWMALMTFTLFYTDFVGEGLYQGVPRAEPGTEARRHYDEGIRMGSLGLFLQCAISLVFSLVMDRLVQKFGTRSVYLASVMTFPVAAAATCLSHSVVVVTASAALTGFTFSALQILPYTLASLYHREKQVFLPKYRGDAGGSSGEDSQTTSFLPGPKPGALFPNGHVGSGSSGILAPPPALCGASACDVSMRVVVGEPPEARVVTGRGICLDLAILDSAFLLSQVAPSLFMGSIVQLSHSVTAYMVSAAGLGLVAIYFATQVVFDKNDLAKYSV

SEQ ID NO: 11 - DNA, 2188 bp, Mus musculus
gagatttaaaaggcgcccgctggcgcgcgttggtgamscaggbgtcgccgagctcgcacgcgccagccccaggtgacagccgcacgccgggccaggatctgaccgacgagatgtgtccccatcaagcaaggcactagatggtgacgtgtttagcgtgggacgagatgctgaattggcactaaagggctggcagaaatgggaacctggctgcaccctaggaggttagtgctagtgaggaggagaagccacggcagggctgactcaaagcagctgtggagtatgtgagtagcccctggaaccctacctgccctgtccatcatgatccagaggctgtgggccagccgtctgctacggcaccggaaagctcagctcctgctggtcaacctgctcacctttggcctggaggtgtgcctggctgccggcattacctatgtgccaccccttctgctggaagtcggggtggaggagaaattcatgaccatggtgttgggcattggcccagtgctaggcctggtttctgttccactcctaggctcagccagtgaccagtggcgtgggcgctatggccgccggagaccctttatctgggctttgtccctgggtgtcctgctaagcctctttctcatcccgagggctggctggctggcaggactgctgtacccagacaccaggcccctggagttggccctgctgatcttgggagtggggctgctggacttttgtggccaggtgtgctttactccattggaggccttactctccgacctcttccgggacccagaccactgccgccaagccttctctgtctacgccttcatgatcagccttgggggctgcctgggctacctcttacctgccattgactgggacaccagcgttctggccccctacctgggtactcaggaagaatgcctctttggcctcctcaccctcattttcctcatctgcatggcagccactctgtttgtgacggaggaggcagtactgggcccacccgagccggcagaagggttgttggtctctgccgtgtcgcgccgatgctgcccatgccacgttggcctggctttccggaatctgggtaccctgtttccccggctgcagcagctgtgctgccgcatgcctcgcaccctacgccgactctttgtggctgagctgtgcagctggatggcacttatgactttcacactgttctacacggacttcgtgggagaggggctgtaccagggtgtacccagagccgagccaggcaccgaggcccggagacactatgatgaaggcattcgaatgggcagcctggggctcttcctgcagtgtgccatctccctggtcttctccctggtcatggacaggctggtacagaagttcggcacacggtcagtctatctggccagtgtgatgacctttcctgtggctgccgctgccacctgcctgtcccacagcgtggtggtagtgacagcctcagctgccctcaccgggttcaccttctcggccttgcagatcctgccttacacgctcgcctccctctaccaccgtgagaagcaggtgttcctgcccaaataccgaggggacgctggaggtagcagcggtgaggacagccagacaaccagcttcttgccaggccctaagccaggagctctcttccccaatggacacgtgggctctggcagcagcggcatcctggcccctccacctgcactctgtggggcctctgcctgcgatgtctccatgcgagtggtggtgggtgagccacctgaggccagggttgttacgggacggggcatttgcctggacctggccattctggacagtgcctttctgctgtcccaggtggctccgtccctgttcatgggctccattgtccagctgagccactctgtcactgcctatatggtatcagctgcaggcttgggtctggtcgccatttactttgctacacaggtagtgtttgacaagaacgacttggccaaatactcagtgtagaattgtgtaaggcatcaaagagagggtctgcctcatgggttctcagcccttagagggctgcagagctggcctctccaggtctttgtcgcctaagtggctctctgctgccaccctaaggcagtgaggtgtattgttgcacagataggagccagagctttcggggctctggcttcagagtctggctggcctactggcagcctctcgcatg